(19) United States
(12) Patent Application Publication
(10) Pub. No.: US 2011/0312497 A1
(43) Pub. Date: Dec. 22, 2011

(54) USE OF HYDROPHOBIN AS A SPREADING AGENT

(75) Inventors: Heiko Barg, Weinheim (DE); Thomas Subkowski, Ladenburg (DE); Marvin Karos, Schwetzingen (DE); Claus Bollschweiller, Heidelberg (DE)

(73) Assignee: BASF SE, Ludwigshafen (DE)

(21) Appl. No.: 13/148,325

(22) PCT Filed: Feb. 10, 2010
(86) PCT No.: PCT/EP2010/051659

§ 371 Date: Aug. 8, 2011

(30) Foreign Application Priority Data
Feb. 10, 2009 (EP) 09001876.3

(51) Int. Cl. A61K 47/42; C07K 14/37; A01N 25/00

(52) U.S. Cl. 504/358; 514/773; 530/350

(57) ABSTRACT

The present invention relates to the use of hydrophobin as a spreading agent, in particular in cosmetic or pharmaceutical compositions. The invention further relates to compositions for treating surfaces, in particular cosmetic or pharmaceutical compositions for topical use, that contain hydrophobin as a spreading agent.

The present invention relates to the use of hydrophobin as a spreading agent, in particular in cosmetic or pharmaceutical compositions. It also relates to compositions for treating surfaces, in particular cosmetic or pharmaceutical compositions for topical application which comprise hydrophobin as a spreading agent.

### BACKGROUND OF THE INVENTION

The spreadability (synonymous with: spread(ing) capacity) describes the ability of substances or substance mixtures (e.g. oils and creams) to spontaneously spread out on a solid, e.g. the skin, and, in so doing, to form a thin film. This spreading on the surface is dependent on the viscosity and surface tension of the substances, the occurrence of surface-active substances, and the properties of the surface (such as e.g. the moisture content of the skin and the sebum content of the horny layer). Good spreading capacity is advantageous e.g. in skin care compositions.

Spreading agents is the term used to refer to substances, the addition of which to a liquid or semi-solid composition, leads to an enlargement of the surface covered by the composition. Spreading agents act by e.g. reducing the surface tension of a composition or by increasing the wettability of the treated surface. As a result, they facilitate the uniform distribution of the composition and its ingredients, and also the formation of a uniform film on the treated surface. The spreading agent suppresses the tendency of compositions with a high surface tension (such as e.g. aqueous solutions) from forming droplets on the treated surface which would lead to spot-like concentration of the substances present in the composition (so-called “spotting”).

The spreading of compositions on surfaces, e.g. of cosmetic or pharmaceutical products on the skin, can be measured (e.g. Zeidler, U. (1985) Fette, Seifen, Anstrichmittel 87:403), the measurement result is referred to as spreading value. B. W. Barry and A. J. Grace have also developed a method for determining the spreading capacity (J. Pharm. Sci. 61, 335 (1972)) and C. Beyer developed a model testing system for testing the spreading capacity (Arch. Pharm. [Weinh.] 310, 473, 729 and 858 (1977); C.A. 88, 79017 (1978); Zbl. Pharm. 118, 51 (1979)).

As spreading tension, the attempt of a liquid to spread out on a solid surface is expressed quantitatively by the difference between the wetting tension and the surface tension of the liquid. This parameter is referred to in accordance with DIN 53900 as spreading coefficient. If this value is positive, then the liquid spontaneously extends (spreads) out on the surface.

According to DIN 53900, spreading capacity is the ability of a substance to spontaneously cover a surface of a liquid or else of a solid. Spreading capacity is therefore understood in cosmetics as meaning the property of a substance to spread on the skin. The measurement unit of the spreading coefficient is that of the quotient of the spreading area over which the spreading takes place, and the spreading time in which the spreading takes place. It is usually given in [mm²/minutes].

Spreading agents are needed in a multitude of products, inter alia in crop protection compositions, compositions for the care of technical surfaces, cosmetics and drugs.

Crop protection compositions are usually sprayed onto the treated plants and must spread as rapidly and uniformly as possible on the plants in order to ensure rapid and uniform distribution of the active ingredient before it can be rinsed off e.g. by rain.

In the cosmetics and pharmaceutical sector, the spreading properties of emulsions, creams, ointments, gels, pastes, body milk and oils are primarily of interest. Cosmetic and pharmaceutical compositions are often O/W or W/O emulsions. The spreading properties of emulsions are usually determined by their oil component(s) (synonymous with: oil body).

Spreading agents using the example of compositions for treating the skin are explained in more detail below. These explanations can also be applied analogously to spreading agents in compositions for other technical fields such as e.g. crop protection.

Spreading agents can be divided into low-spreading (spreading value: <300 mm²/10 min), medium-spreading (ca. 300-1000 mm²/10 min) and high-spreading spreading agents (>1000 mm²/10 min). This describes not only their behavior on the skin as regards their spreadability, but also the intensity and duration of their smoothing effect. Spreading agents are mostly oils or fats; for this reason, these will be explained in more detail below by way of example.

Rapidly spreading oils spread very readily, penetrate rapidly into the indentations in the surface of the skin and produce a rapid feeling of smoothness, although this likewise rapidly returns again to the old level.

Rapid spreaders are primarily oils with many short-chain fatty acids (C6 to C10), e.g. coconut oil and babassu oil, but also plant butters. In addition, squalane is a very rapidly spreading fatty component.

Medium-spreading oils spread well, exhibit a pleasant absorption behavior and confer smoothness over a significantly longer period. These are oils with medium-length fatty acid chains (C12 to C16) or with high lecithin or squalene content e.g. avocado oil, sesame oil, grapeseed oil or amaranth oil. The accompanying substances of these oils alter their surface tension (lecithins belong e.g. to the phospholipids and are interface-active lipids) and thereby bring about more rapid spreading on the skin (compared to their spreading behavior which is to be expected on account of their fatty acid spectrum) and better absorption.

Slow-spreading oils lead to a significantly less marked feeling of smoothness, although this lasts for a long time. This group includes in particular oils with long-chain fatty acids (C18 to C24), e.g. evening primrose oil, borage oil, hemp oil and wild rose oil.

In order to optimize a formulation, oils or other spreading agents can be combined with one another or with other substances, according to their spreading behavior. Thus, an emulsion which comprises rapidly-spreading, medium-spreading and slow-spreading oils alongside one another ensures a more pleasant application behavior and skin feel than do emulsions with only one oil. This combination is also referred to as spreading cascade. Formulations which do not take into consideration spreading agents of all three categories generally have a spreading gap, i.e. they are lacking a component with a specific effect which determines the duration or rapidity of the feeling of smoothness.

It is obvious that the development of spreading cascades is associated with complexity and is therefore in need of improvement. Moreover, spreading in the case of such multicomponent systems is difficult to dose and predict. Added to this is the fact that many oils and fats used as a spreading agents leave behind an undesired shiny film on the skin or can even lead to undesired sealing of the skin. However, consumers desire products which convey a rapid-onset and long-lasting feel of skin smoothness and suppleness and at the same time do not leave behind either a sticky, greasy feel or a greasy shimmering film on the skin. It can also lead to undesired incompatibility reactions between the spreading agent and other components of the composition or of the treated surface.

On the other hand, the complete omission of a spreading agent, particularly in the case of aqueous solutions, leads to poor spreading behavior and its indirect consequences, such as e.g. spotting. The use of oils as a spreading agents in aqueous systems, however, is often undesired or, in the event of lack of miscibility of an oil with water, is possible only with the addition of emulsifiers.

Under the circumstances, there is the need for a spreading agent which does not have the disadvantages as oils as a spreading agent and can replace these in part or completely in a composition and in particular is miscible with water. This is true not only for cosmetic or pharmaceutical compositions, but also for non-cosmetic/non-pharmaceutical preparations, such as e.g. polishes, shoe cream, crop protection compositions etc. In all of these fields of application, there is the need for compositions which do not “spot” and do not leave behind greasy residues.

Hydrophobins are a class of small, cysteine-rich proteins with a length of about 100-150 amino acids which occur in nature only in filamentous fungi. They are amphiphilic and can form a water-insoluble layer on the surface of an object. Their natural functions include inter alia the coating of fungal spores, so that these do not stick together, the coating of aerial hyphae for reducing the surface tension of water and thus for facilitating the absorption of water, and possibly the signal transmittance between a fungus and its environment (Whiteford, J. F. Spanu, P. D. (2001), Fungal Genet. Biol. 32 (3): 159-168; Wösten et al. (1999) Current Biol. 19: 1985-88; Bell et al. (1992), Genes Dev. 6: 2382-2394).

The first isolation and purification of hydrophobin was carried out from Schizophyllum commune in 1999. In the meantime, hydrophobin genes have been identified in Ascomycetes, Deuteromycetes and Basiodiomycetes. Some fungi comprise more than one hydrophobin gene, e.g. Schizophyllum commune, Coprinus cinereus and Aspergillus nidulans.

On the basis of differences with regard to the hydropathy and the biophysical properties of the hydrophobins, these have been divided into two categories: class I and class II. Complementation experiments have shown that hydrophobins of the one class are able to replace hydrophobins of the other class to a certain degree as far as function is concerned. The different hydrophobins appear to be involved in different fungal development stages and to perform different functions therein (van Wetter et al. (2000) Mol. Microbiol. 36:201-210; Kershaw et al. (1998) Fungal Genet. Biol. 23:18-33).

Hydrophobins generally have eight cysteine units. They can be isolated from natural sources, but can also be obtained by means of genetic engineering methods, as described for example in WO 2006/082251 and WO 2006/131564.

The use of hydrophobin in cosmetic preparations is known per se. US 2003/0217419 A1 describes the use of the hydrophobin SC3 from S. commune for the treatment of keratin-containing materials. Here, cosmetic depots are formed which are supposed to withstand several washes with shampoo. The hydrophobin is applied either at the same time as or after the cosmetically active ingredient, but not before applying the cosmetic active ingredient.

WO 2006/136607 A2 describes the use of hydrophobin and of hydrophobin conjugates in cosmetic preparations for hair care.

WO 2006/082251 describes hydrophobin proteins, their production and their use for the surface coating of glass and Teflon.

WO 96/41882 proposes the use of hydrophobins inter alia as surface-active substances, for the hydrophilization of hydrophobic surfaces, for improving the water resistance of hydrophilic substrates and for producing emulsions, ointments, creams, hair shampoos and rinses.

### AIM OF THE INVENTION

The object of the present invention is to provide spreading agents for compositions for surface treatment, in particular for cosmetic and pharmaceutical compositions that are to be applied topically.

A further object consists in providing compositions in which oils and fats as a spreading agents are replaced partially or completely by at least one other spreading agent.

A further aim is the provision of non-greasy compositions, in particular aqueous compositions, with good spreading properties.

The spreading agent according to the invention should be able to be used on a large number of surfaces, primarily on natural surfaces, such as human and animal skin or plant surfaces.

These and other aims as arise from the following description of the invention are achieved by the present invention according to the independent claims. Particular embodiments of the invention can be found in the dependent claims, the description and the examples. Furthermore, the invention also comprises combinations of these preferred embodiments.

### SHORT DESCRIPTION OF THE INVENTION

The present invention relates to the use of hydrophobin as a spreading agent. It furthermore relates to a composition which comprises hydrophobin as a spreading agent.

Good spreading behavior of a composition is advantageous if the composition is to be spread evenly on a surface. This requirement is met by the uses and compositions according to the invention.

According to the invention, hydrophobin can be used as a spreading agent in compositions for treating a very wide variety of surfaces, such as e.g. for treating technical surfaces (paint, metal, plastic) and natural surfaces (skin, hair, teeth, nails, leather, textiles, wool, plant surfaces). It preferably serves as a spreading agent in a cosmetic or pharmaceutical composition or in a crop protection composition.

The use of hydrophobin preferably leads to the oil content required for the spreading in the compositions being able to be reduced compared to compositions without hydrophobin, for example by from 50% up to 100%. According to the invention, hydrophobin can thus serve as partial or complete oil body replacement. A further effect is that, as a result, shining of the treated surface such as e.g. the skin is reduced or prevented because less or even no oil or grease is applied to the surface.

The use of hydrophobin can furthermore lead to an improved absorption and/or accumulation of other constituents of the hydrophobin-containing composition. It preferably brings about improved absorption and/or accumulation of one or more active ingredients, e.g. of cosmetic and/or pharmaceutical active ingredients, in/on the tissue treated with the hydrophobin-containing composition, such as, for example, the skin. This relates in particular to hydrophilic substances such as cosmetically or pharmaceutically active ingredients, e.g. panthenol. As a result, the active ingredients have a more intensive action since they pass more quickly, more uniformly and/or in a higher local concentration onto and/or into the treated surface, and/or the time until the substances are completely washed is extended. The absorption and distribution of crop protection active ingredients can also be improved by hydrophobin as a spreading agent.

Specifically, the present invention relates to the following subjects and embodiments:
(1) the use of hydrophobin, e.g. of hydrophobin of the structural formula (I) as a spreading agent in a composition and/or of a composition for treating surfaces;
(2) the use of hydrophobin, e.g. of hydrophobin of the structural formula (I) as a spreading agent according to embodiment (1) in a cosmetic or pharmaceutical composition, in particular a composition for treating human or animal skin;
(3) the use according to embodiment (1) or (2), where additionally at least one active ingredient is applied to the treated surface, the absorption of which into the surface and/or the effect of which on the surface is improved by the presence of hydrophobin; and
(4) a composition for surface treatment which comprises at least one hydrophobin, e.g. hydrophobin of the structural formula (I), and is preferably a cosmetic or pharmaceutical composition or a crop protection composition.

### DEFINITIONS

The following terms, definitions and abbreviations are used: customary three- and one-letter codes for amino acids and nucleotides.

Within the context of the present invention, the singular form “a” also includes the respective plural unless the context gives rise to something different. The term “a hydrophobin” can thus also comprise more than one hydrophobin molecule, namely two, three, four, five etc. hydrophobins of one sort.

“At least one” means “one or more”, “at least” followed by a numerical value means “this or a higher numerical value”.

The term “about” or “ca.” in connection with a numerical value or a parameter range limit refers to an uncertainty range in which, in the view of the person skilled in the art, the technical effect of the feature in question is still assured. The term typically means a deviation from the stated numerical value of +/−10%, preferably +/−5%.

Unless stated otherwise, acids are present either as free acid or as partial or complete salt of the acid or as a mixture of the acid with its salt. Conversely, bases, in particular amines, can be present as free base or as partial or complete salt of the base or as a mixture of the base with its salt.

“Native” is synonymous with “wild type” and “naturally (occurring)”. A “naturally” occurring linkage of two polypeptides is a linkage as is found in naturam, i.e. e.g. in a wild type protein. A wild type or native protein or polypeptide is—unless stated otherwise—the customary naturally occurring form of this protein/polypeptide.

Within the context of the present invention, “recombinant” means “produced with the help or the result of genetic engineering methods”.

A “fragment” of an amino acid sequence results from the lack of one or more successive amino acids on the N and/or C terminus of the specified original sequence.

Within the context of the present invention, a “homolog” of an amino acid sequence is a protein or polypeptide which differs from the original sequence by virtue of the substitution of one or more amino acids. Preferably, the function and/or conformation of the protein is not impaired by this substitution. The amino acid substitution is particularly preferably a conservative amino acid exchange, the exchanged amino acids are thus replaced by amino acids with similar chemical properties, e.g. Val by Ala.

Conservative amino acid exchanges take place particularly preferably between the members of the following groups:
acidic amino acids (asparate and glutamic acid);
basic amino acids (lysine, arginine, histidine);
hydrophobic amino acids (leucine, isoleucine, methionine, valine, alanine);
hydrophilic amino acids (serine, glycine, alanine, threonine);
amino acids with aliphatic side chains (glycine, alanine, valine, leucine, isoleucine);
amino acids with hydroxyaliphatic side chains (serine, threonine);
amino acids with amide groups in the side chain (asparagine, glutamine);
amino acids with aromatic side chains (phenylalanine, tyrosine, tryptophan);
amino acids with sulfur in the side chain (cysteine, methionine).

Specifically preferred conservative amino acid exchanges are:

| Original amino acid | Substitute |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term “isolated” means “separated or purified from the original organism”. An isolated hydrophobin is consequently no longer a constituent of the fungus in which it occurs naturally. Hydrophobins produced by recombinant means are also “isolated” hydrophobins.

The terms “hydrophilic” and “hydrophobic” have the meaning customary in chemical terminology. A “hydrophilic” substance is thus a substance which is preferably soluble in water and/or polar solvents. Hydrophilic substances are typically polar compounds which are either ionic, have a dipole moment and/or comprise electronegative groups. A “hydrophobic” substance, on the other hand, preferentially dissolves in nonpolar media and has no ionic functional groups and only weakly electronegative functional groups.

The term “non-greasy” has the meaning customary for pharmaceuticals and cosmetics in the specialist terminology. A “non-greasy” cream as a rule leaves behind no layer of grease or oil and no sticky feel on the skin.

### DETAILED DESCRIPTION OF THE INVENTION

The present invention relates to the use of hydrophobin as a spreading agent and to corresponding hydrophobin-comprising compositions.

The formulation of products comprising oil bodies as a spreading agents, in particular of cosmetics, pharmaceuticals and crop protection compositions, is prior art. In the present invention, the object was to develop formulations which, despite a reduced content of oil bodies, have very good properties, in particular have good spreadability. As regards cosmetic and pharmaceutical compositions, the use according to the invention of hydrophobin as a spreading agent preferably also brings about good sensory properties, in particular silky skin feel, high glidability after the product has soaked onto the skin, and also low stickiness.

The fact that hydrophobin can serve as a spreading agent instead of customary oil bodies was surprising since hydrophobin is a protein and proteins are not usually used as a spreading agents.

In the case of the use according to embodiment (1), the composition has an oil-body concentration which is lower than the oil-body concentration in a composition of the same ingredients but without hydrophobin and with a higher oil-body fraction if both compositions have the same spreading behavior. Preferably, in the case of the use according to embodiment (1), the composition has an oil-body concentration of from 0 to 20% by weight, particularly preferably from 0 to 10% by weight, very particularly preferably from 0 to 5% by weight.

The spreading agent has a wetting function and brings about optimal distribution of the other components over a large area. As a result, this means that partial superconcentrations do not result on the treated surface which would be visible as marks (spotting).

The addition of hydrophobin as a spreading agent moreover leads to improved spreadability of a composition on the surface, e.g. of a cosmetic composition on the skin. This spreadability is essential particularly for a preparation containing active ingredient in order to ensure a good uniform distribution of the active ingredients on the surface, e.g. the skin. A criterion for good distributability here is the spreading capacity of the hydrophobin.

The composition present in all embodiments of the invention which comprises hydrophobin as a spreading agent is generally a liquid or semi-solid composition. “Semi-solid compositions” here have those properties which are ascribed to them in the definition given in the European Pharmacopoeia (Ph. Eur., 6th edition). However, the compositions used in the course of the invention are not exclusively pharmaceutical or cosmetic compositions. All compositions which serve for the treatment of surfaces are in accordance with the invention. However, the hydrophobin is preferably used in a pharmaceutical or cosmetic composition as a spreading agent.

A composition according to the invention can be an emulsion, a cream, a gel, a paste, an ointment, a body milk, a lotion, a foam, a suspension, a milk or a paste. The skin care compositions according to the invention are in particular W/O or O/W emulsions. Specifically, skin care compositions according to the invention are selected from the group consisting of skin creams, day and night creams, eye creams, face creams, antiwrinkle creams, sun protection creams, moisturizing creams, bleaching creams, self-tanning creams, vitamin creams, skin lotions, care lotions and moisturizing lotions. A solution as is used e.g. in a spray is also possible. In this connection, preference is given to an aqueous solution. Solutions are preferred particularly when using hydrophobin as a spreading agent in a crop protection composition.

In one preferred aspect, the composition according to the invention in which hydrophobin is used as a spreading agent is a hydrophilic composition, in particular a solution, a hydrophilic ointment or hydrophilic cream.

For the use according to the invention, the hydrophobin is advantageously present in a composition which preferably also comprises an acceptable carrier medium, depending on the field of application e.g. a cosmetically acceptable, pharmaceutically acceptable or crop-protection acceptable carrier medium. The composition according to the invention as in embodiment (4) in which the hydrophobin is used as a spreading agent according to one of the embodiments (1) to (3), is, in one aspect of the present invention, a composition in which the carrier medium is water, one or more hydrophilic solvents, or a mixture of water and one or more hydrophilic solvents. The fraction of water and/or hydrophilic solvents in the composition is preferably more than 40% by weight, particularly preferably more than 50% by weight, very particularly preferably more than 60% by weight, 70% by weight, 80% by weight, 90% by weight or 95% by weight. It is particularly preferred that exclusively water and/or hydrophilic solvents are used as carrier medium. The only carrier medium is very particularly preferably water.

Spreading compositions such as creams, lotions or milks, e.g. bodycare products, usually comprise oil bodies (synonymous with: oil component), which contribute to optimizing the spreading behavior. These oil bodies are usually present in conventional compositions in a total amount of more than 50% by weight, in most cases from 5 to 50% by weight, predominantly from 5 to 25% by weight.

In a preferred aspect of the invention, the hydrophobin is used according to the invention in compositions which comprise little or no grease or oil, i.e. few or no oil components. Oil components are in particular oils, fats, waxes, silicone compounds, specifically hydrophobic lipids such as hydrocarbons, silicone oils, fatty alcohols and fatty acid esters.

Preferably, compositions according to the invention with hydrophobin as a spreading agent comprise less than 50% by weight of oil component, particularly preferably less than 40% by weight, very particularly preferably less than 25% by weight, 20% by weight, 15% by weight, 10% by weight, in particular less than 5% by weight, and most preferably 0% by weight, of oil component. In one preferred embodiment, the hydrophobin is used as a spreading agent in a composition which is free from oil components.

In a further preferred embodiment, the hydrophobin is used in a composition which comprises less than 5% by weight of emulsifiers and is particularly preferably free from emulsifiers. As a result of this, one disadvantage of emulsifiers is avoided: the undesired washing-out of fats from the skin which arises when cleaning the skin by emulsifiers, which are retained on or in the skin.

Very particular preference is given to one embodiment in which neither emulsifiers nor oil components are used for the compositions according to the invention.

In one aspect of the present invention, the hydrophobin is used as a spreading agent in a non-greasy composition, particularly in a non-greasy cream or lotion, such as e.g. a skin cream.

Compositions with hydrophobin as a spreading agent manage with a minimum of oil component and require no further auxiliaries for emulsion formation or for improving the spreading behavior. This is very accommodating for sensitive skin in particular.

The subject matter of the present invention serves for the treatment of surfaces. The composition according to the invention as in embodiment (4), in which the hydrophobin according to one of the embodiments (1) to (3) is used as a spreading agent, is a composition for treating surfaces. Surfaces to be treated according to the invention are all surfaces on which the spreading out of the composition for surface treatment following application to the surface is desired. This includes technical and natural surfaces.

Technical surfaces which can be treated according to the invention include surfaces made of plastic, paper, metal, paint, textiles, leather or other natural substances, in particular keratin-containing textiles, leather and paper. Consequently, compositions according to the invention for surface treatment are e.g. also automobile polishes, cleaners, impregnating compositions, compositions for paper treatment and shoe cream.

Natural surfaces which can be treated according to the invention include plant and animal materials, in particular plant, human or animal body surfaces.

In one aspect, the preferred natural surfaces are human or animal body surfaces. These are preferably keratin-containing surfaces, such as skin, hair, nails, horn, wool, leather, hide, fur and feathers. Particular preference is given to hair and skin. Very particularly preferably, hydrophobin is used according to the invention as a spreading agent in a composition for treating skin and the composition as in embodiment (4) is suitable for treating the skin. Human skin is preferred.

In a second aspect, the preferred natural surfaces are made of plant material, in particular plant surfaces as are wetted by these compositions when applying crop protection compositions. In a further preferred aspect, the preferred treated surface made of plant material is a cellulose-containing surface, in particular paper.

A further aspect of the present invention is the use of hydrophobin for producing one of the specified compositions for treating surfaces, in particular a crop protection composition, a cosmetic or a drug.

For applying the hydrophobin-containing compositions, in principle all methods and techniques available for the targeted application of a liquid or semi-solid medium to a technical or natural surface are suitable. In the case of technical surfaces, these are, for example, immersion, printing, painting or spraying methods and also application by means of brushes, knives or grooved rollers. In the case of natural surfaces, these are in particular spreading (rubbing in), lathering, spraying and painting. The technique to be selected for applying the composition is governed by the type of nature of the surface to be coated, and/or the shape of the body having the surface. For treating e.g. metal or paper, customary painting methods can be used, whereas in the case of curved shapes, spraying techniques are more suitable. For treating e.g. skin, rubbing in or spraying is preferred, whereas the treatment of hair preferably takes place by lathering or spraying.

The use according to embodiment (1) to (3) and the surface treatment with the composition according to embodiment (4) of the present invention require the presence of at least one hydrophobin in a composition which serves for the surface treatment. This composition preferably also comprises further ingredients, in particular at least one substance which serves for the care of the treated surface, such as e.g. panthenol, vitamins or bisabolol in the case of cosmetic and pharmaceutical skin care or hair care products. In the composition according to the invention comprising hydrophobin as a spreading agent, a large number of different active ingredients and effect substances can be formulated. If the composition is a cosmetic or pharmaceutical composition, then it preferably comprises, besides hydrophobin, further pharmaceutically or cosmetically acceptable ingredients. Details of these ingredients are discussed below.

Within the context of all embodiments of the present invention, “hydrophobin” or “hydrophobins” are preferably understood as meaning polypeptides of the general formula (I)

Xₙ-C¹—X₁₋₅₀—C²—X₀₋₅—C³—X₁₋₁₀₀—C⁴—X₁₋₁₀₀—C⁵—X₁₋₅₀—C⁶—X₀₋₅—C⁷—X₁₋₅₀—C⁸—Xₘ (I)

where X can be any of the 20 naturally occurring amino acids (Phe, Leu, Ser, Tyr, Cys, Trp, Pro, His, Gln, Arg, Ile, Met, Thr, Asn, Lys, Val, Ala, Asp, Glu, Gly). In this connection, the radicals X may in each case be identical or different. Here, the indices alongside X are in each case the number of amino acids in the respective part sequence X.

The indices n and m are, independently of one another, natural numbers. In general, neither m nor n are zero, but are generally 1 or more. For example, m and n, independently of one another, can be from 1 to 500. Preferably, m and n, independently of one another, are from 15 to 300.

The amino acids designated C¹ to C⁸ are preferably cysteines; however, they can also be replaced by other amino acids of similar spatial filling, preferably by alanine, serine, threonine, methionine or glycine. However, at least four, preferably at least five, particularly preferably at least six and in particular at least seven, of the positions C¹ to C⁸ should be occupied by cysteine.

Cysteines at positions C¹ to C⁸ in the proteins according to the invention can either be present in reduced form or form disulfide bridges with one another. Preference is given to the intramolecular formation of C—C bridges, in particular the formation of at least one, preferably two, particularly preferably three and very particularly preferably four, intramolecular disulfide bridges. In the case of the above-described exchange of cysteines by amino acids of similar spatial filling, advantageously those C positions are exchanged in pairs which are able to form intramolecular disulfide bridges with one another in the case of the presence of Cys at the positions in question.

If a cysteine, serine, alanine, glycine, methionine or threonine is likewise at the positions referred to as X, the numbering of the C positions in the general hydrophobin formulae can change accordingly. Additional cysteines at X positions are likewise able to form disulfide bridges.

Preference is given to using hydrophobins of the general formula (II)

Xₙ-C¹—X₃₋₂₅—C²—X₀₋₂—C³—X₅₋₅₀—C⁴—X₂₋₃₅—C⁵—X₂₋₁₅—C⁶—X₀₋₂—C⁷—X₃₋₃₅—C⁸—Xₘ (II)

for carrying out the present invention, where X and C and the indices alongside X have the above meaning. The indices n and m are natural numbers including the number zero. In general, neither m nor n are zero, but are generally one or more. For example, m and n, independently of one another, can be from 1 to 500. Preferably, m and n, independently of one another, are from 15 to 300. Furthermore, at least six of the radicals designated C are preferably cysteine, and particularly preferably all of the radicals C are cysteine. Very particularly preferably, at least one pair of these cysteines form a disulfide bridge, and the formation of more than one disulfide bridge, i.e. of 2, 3 or 4 of these bridges, is most preferred.

Particular preference is given to using hydrophobins of the general formula (III)

Xₙ-C¹—X₅₋₉—C²—C³—X₁₁₋₃₉—C⁴—X₂₋₂₃—C⁵—X₅₋₉—C⁶—C⁷—X₆₋₁₈—C⁸—Xₘ (III)

for carrying out the present invention, where X and C and the indices alongside X have the above meaning. In particular, the indices n and m are natural numbers from 1 to 200. In general, at least six of the radicals designated C are cysteine. Particularly preferably, all of the radicals C are cysteine. Very particularly preferably, at least one pair of these cysteines form a disulfide bridge, and the formation of more than one disulfide bridge, i.e. of 2, 3 or 4 these bridges, is most preferred.

In each of the formulae (I) to (III), the groups Xₙ and Xₘ are peptide sequences which are naturally linked to the other constituents of the hydrophobin. However, one or both groups may be peptide sequences which are naturally not linked with the other constituents of the hydrophobin. This is also to be understood as meaning those groups Xₙ and/or Xₘ in which a peptide sequence which occurs naturally in the protein is extended by a peptide sequence which does not occur naturally in the protein.

The group Xₙ and/or Xₘ can completely or partially comprise peptide sequences which do not occur naturally in the hydrophobin protein. The peptide sequences not naturally occurring in the protein and from which the group Xₙ and/or Xₘ can partially or completely exist are also referred to below as fusion partners. These fusion partners are generally at least 20, preferably at least 35, amino acids in length. They may be, for example, sequences made of from 20 to 500, preferably from 30 to 400 and particularly preferably from 35 to 100, amino acids.

The fusion partner can be selected from a large number of proteins. Just a single fusion partner may be linked with the radical of the polypeptide, or else two or more fusion partners may be linked with the radical of the polypeptide. For example, it is possible for two fusion partners to be linked with the radical of the polypeptide at one of the positions Xₙ or Xₘ, or for one or more fusion partners to be located at each of the two positions.

Suitable fusion partners are disclosed for example in WO 2006/082251, WO 2006/082253, WO 2006/131564 and WO 2007/014897. These fusion partners are preferred fusion partners within the context of the present invention.

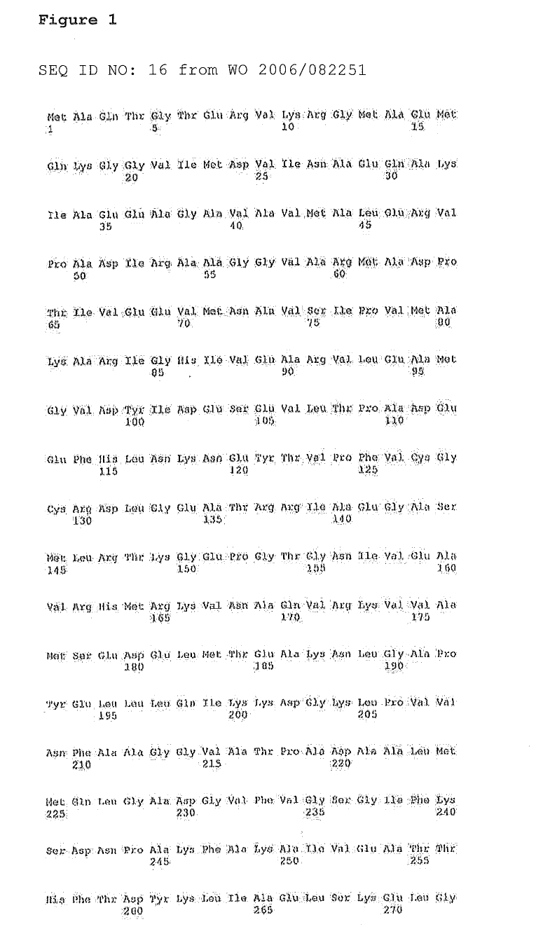

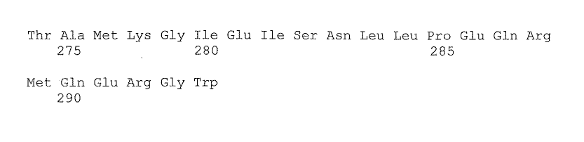

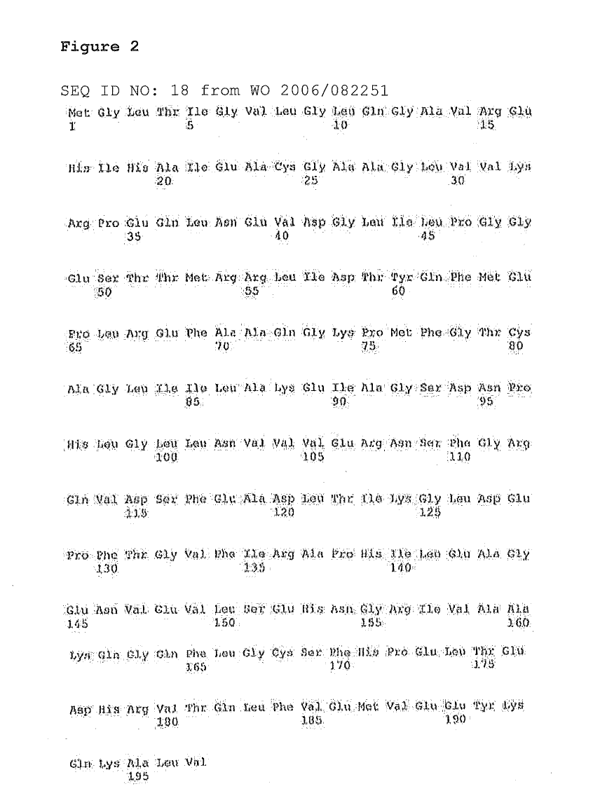

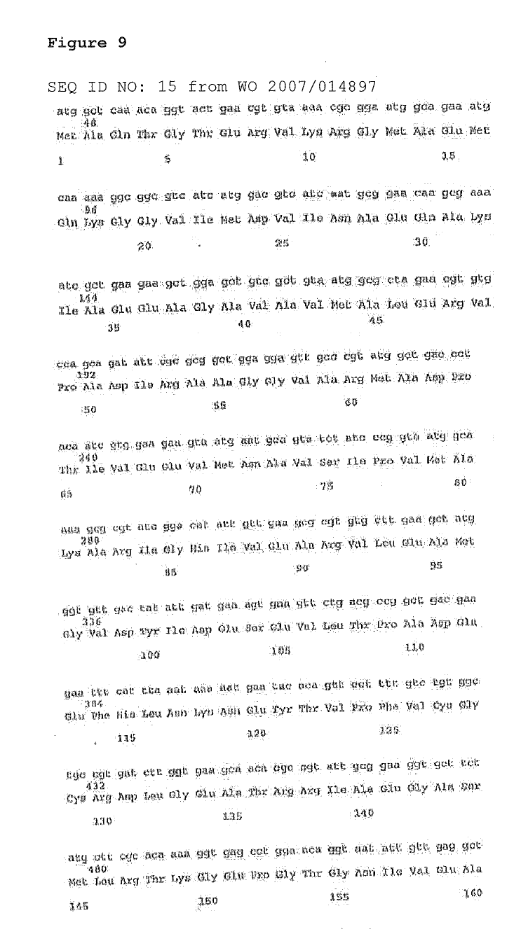

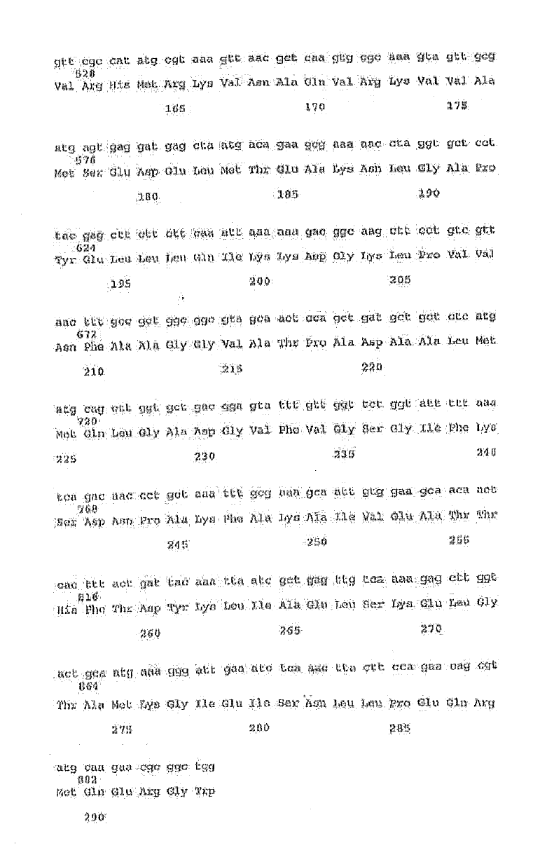

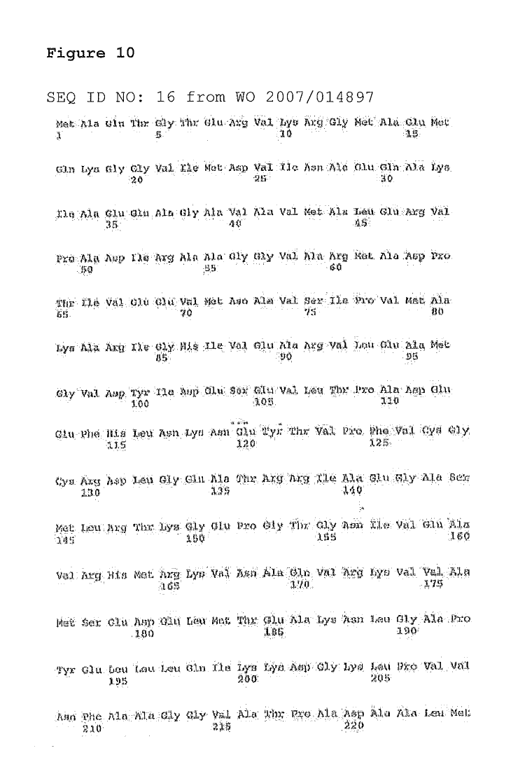

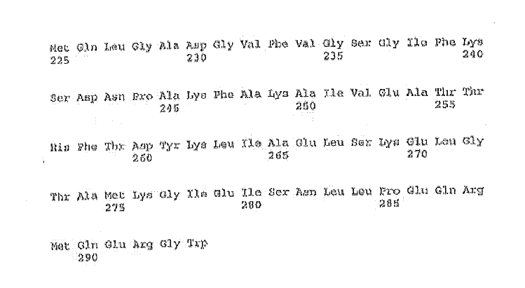

Particularly suitable fusion partners are proteins which occur naturally in microorganisms, preferably in prokaryotes, in particular in Escherichia coli or Bacillus subtilis. Examples of particularly suitable fusion partners are the polypeptides with the sequences yaad (SEQ ID NO:16 in WO 2006/082251, see FIG. 1 of this application; SEQ ID NO:15 or 16 in WO 2007/014897, see FIG. 9 or 10 of this application), yaae (SEQ ID NO:18 in WO 2006/082251, see FIG. 2 of this application), ubiquitin and thioredoxin. Particularly suitable fusion partners are yaad and shortened sequences derived therefrom, as are described in the present description and in WO 2006/082251 and WO 2007/014897. yaad and yaad40 are very particularly suitable. One example of such a protein is yaad40-Xa-dewA-His (SEQ ID NO:26 from WO 2007/014897), which has a yaad radical shortened to 40 amino acids.

Also highly suitable are fragments or homologs of these specified sequences which comprise only one contiguous part, for example from 70 to 99%, preferably from 5 to 50%, and particularly preferably from 10 to 40%, of the amino acids of the specified sequences, or in which individual amino acids, or nucleotides have been exchanged compared with the specified sequence, the percentages referring in each case to the total number of amino acids. Preferred exchanges are described above under “Definitions”.

In a further preferred embodiment, the hydrophobin—optionally preferably alongside one of the fusion partners already mentioned—also has, as one of the groups Xₙ or Xₘ or as terminal constituent of such a group, a so-called affinity domain (affinity tag/affinity tail). In a manner known in principle, these are anchor groups which are able to interact with certain complementary groups and can serve for easier work-up and purification of the proteins. Examples of such affinity domains comprise (His)ₖ, (Arg)ₖ, (Asp)ₖ, (Phe)ₖ or (Cys)ₖ groups, where k is in general a natural number from 1 to 10. Preferably, it may be a (His)ₖ group, where k is one of the numbers four to six. Here, the group Xₙ and/or Xₘ can consist exclusively of such an affinity domain or else of amino acids or polypeptides linked naturally or non-naturally with the radical of the polypeptide and such an affinity domain.

In a further preferred embodiment, the hydrophobin is additionally modified on its polypeptide sequence, for example by glycosylation, acetylation or else by chemical crosslinking, for example with glutardialdehyde.

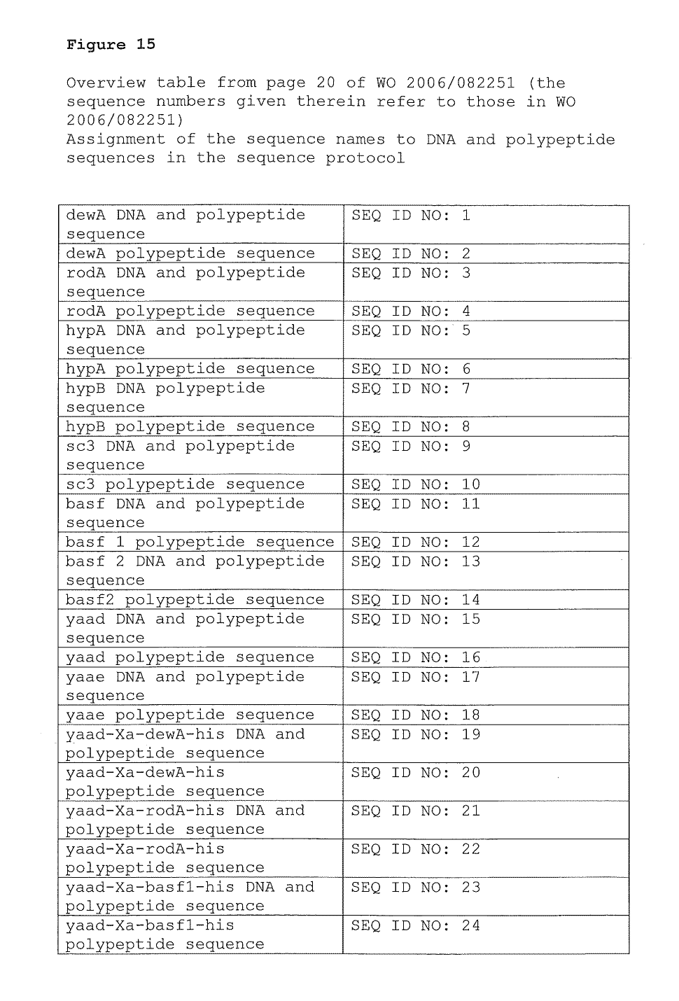

Hydrophobins, their sequences and their preparation are disclosed for example in WO 2006/082251, the contents of which in this regard are hereby expressly incorporated into the present invention. The hydrophobins described in WO 2006/082251 are preferred for carrying out the present invention. Particularly preferred hydrophobins for carrying out the present invention are the hydrophobins of the type dewA, rodA, hypA, hypB, se3, basf1 and basf2, very particularly hydrophobins of the type dewA (present in the examples in the fusion proteins “hydrophobin A” and “hydrophobin B”), hypA and hypB, in particular hydrophobins of the type dewA. These hydrophobins and their sequences are disclosed for example in WO 2006/082251 and WO 2007/014897, the contents of which in this regard are hereby expressly incorporated into the present invention. Unless stated otherwise, the sequence names and SEQ ID numbers given below refer to the sequences disclosed in WO 2006/082251. An overview table with the SEQ ID numbers from WO 2006/082251 can be found in WO 2006/082251 on page 20 (FIG. 15 of this application).

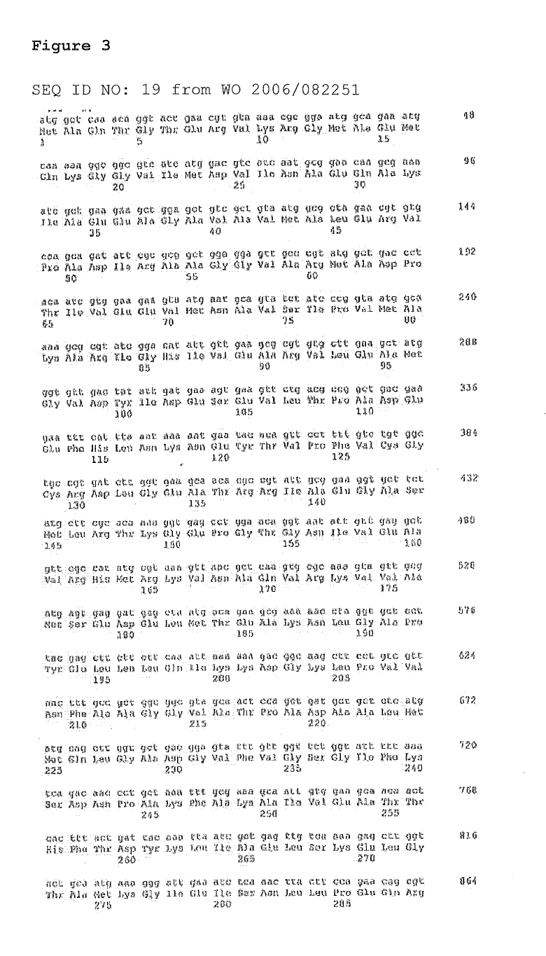

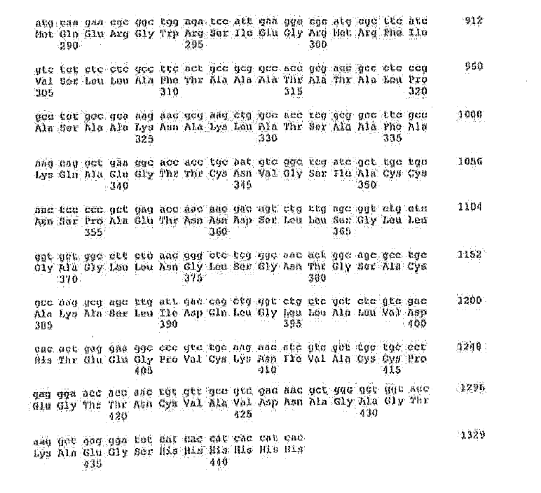

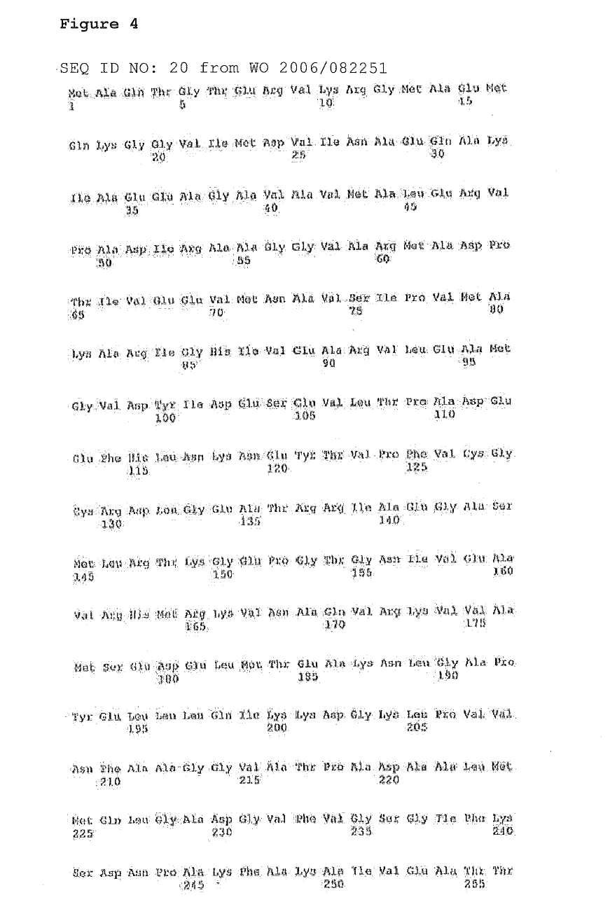

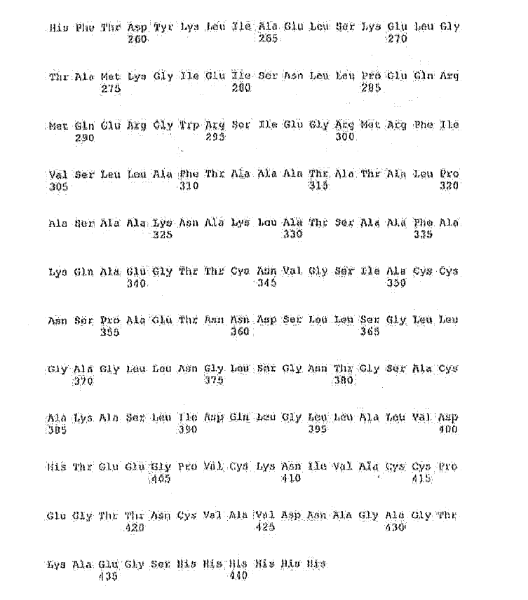

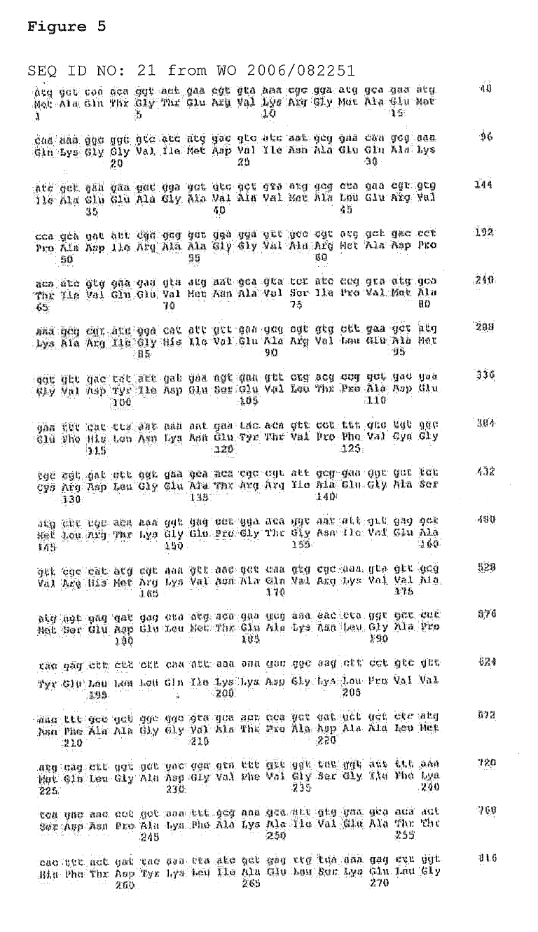

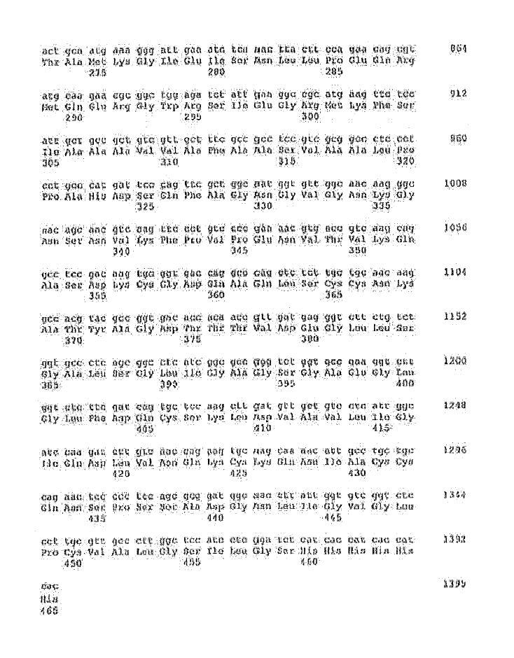

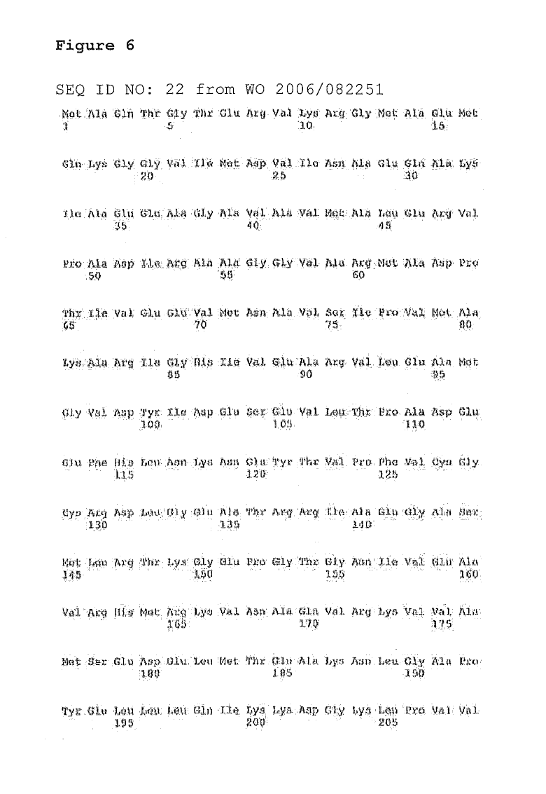

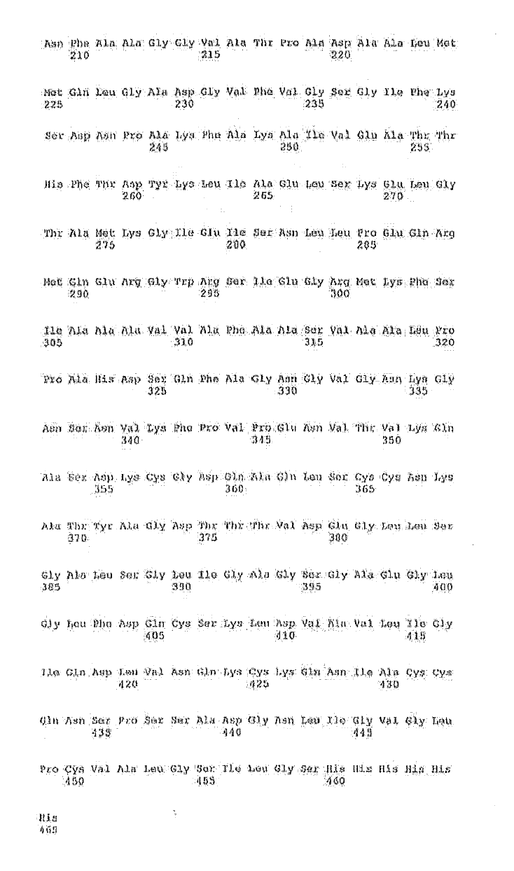

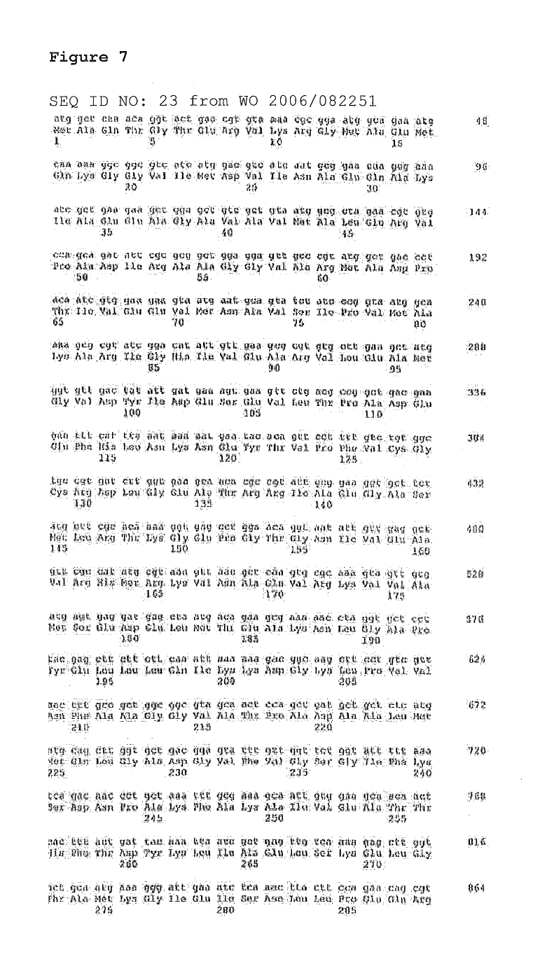

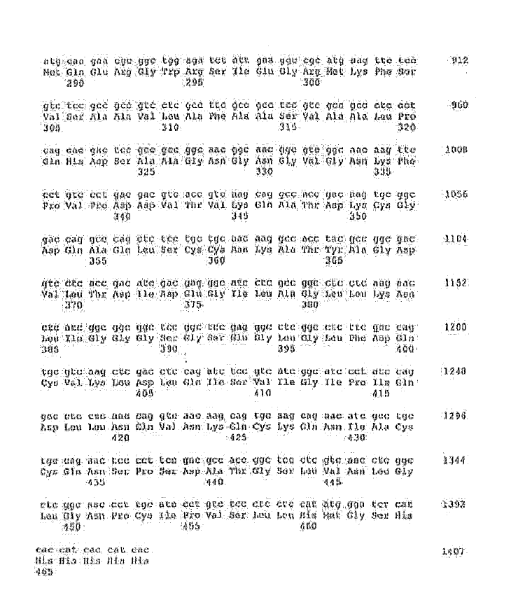

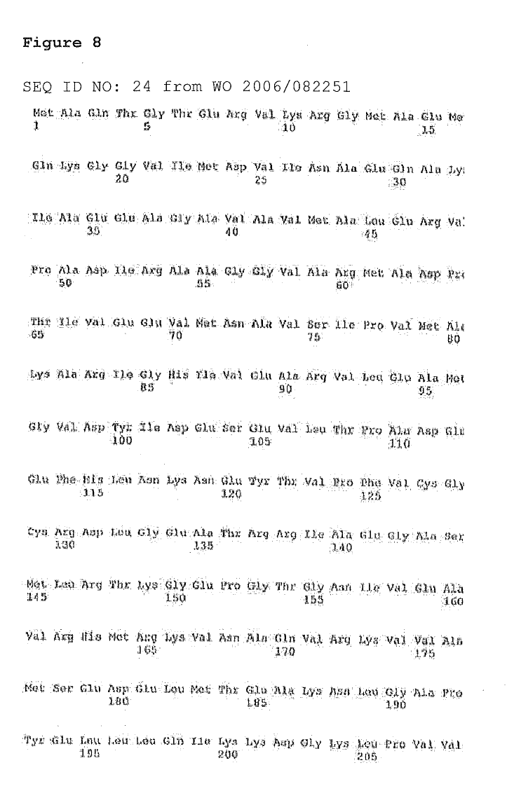

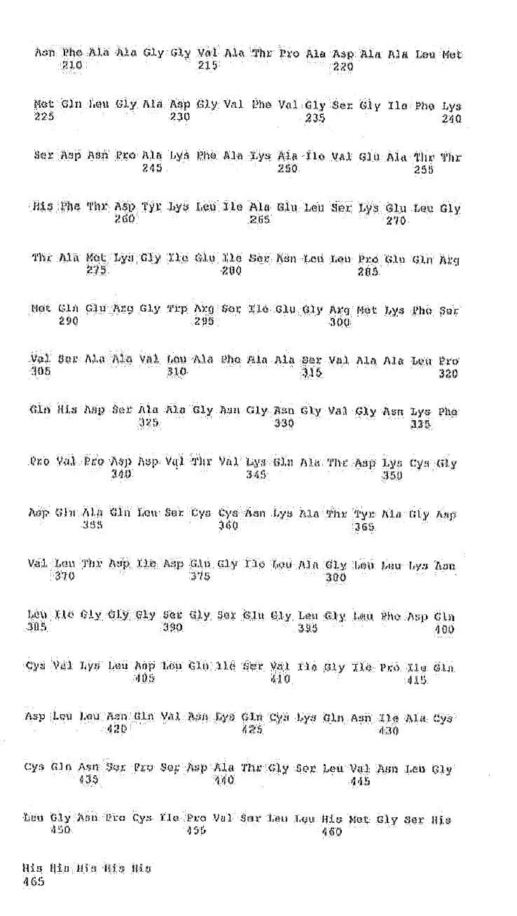

Of particular suitability according to the invention are hydrophobins selected from the group consisting of yaad-Xa-dewA-his (SEQ ID NO:20, FIG. 4 of this application), yaad-Xa-rodA-his (SEQ ID NO:22, FIG. 6 of this application) and yaad-Xa-basf1-his (SEQ ID NO:24, FIG. 8 of this application) with the polypeptide sequences given in brackets, and the nucleic acid sequences coding therefor, in particular, the sequences according to SEQ ID NO:19, 21, 23 (FIGS. 3, 5, 7 of this application). Particularly preferably, yaad-Xa-dewA-his (SEQ ID NO:20 in WO 2006/082251 or SEQ ID NO:19 and 20 in WO 2007/014897, see FIGS. 4 and 11 and 12 of this application) can be used. Also proteins which arise starting from the polypeptide sequences shown in SEQ ID NO:20, 22 or 24 (FIG. 4, 6 or 8 of this application) as a result of exchange, insertion or deletion of at least one, preferably up to 5% of all amino acids, particularly preferably up to ten, very particularly preferably up to five amino acids, and which still have the biological property of the starting proteins to at least 50%, are particularly preferred embodiments.

Biological property of the proteins is understood here as meaning the change, described below, in the contact angle and/or the effect on skin and/or keratin described below.

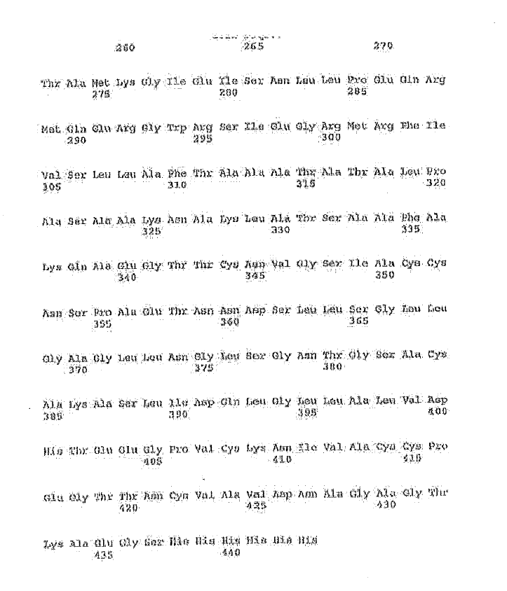

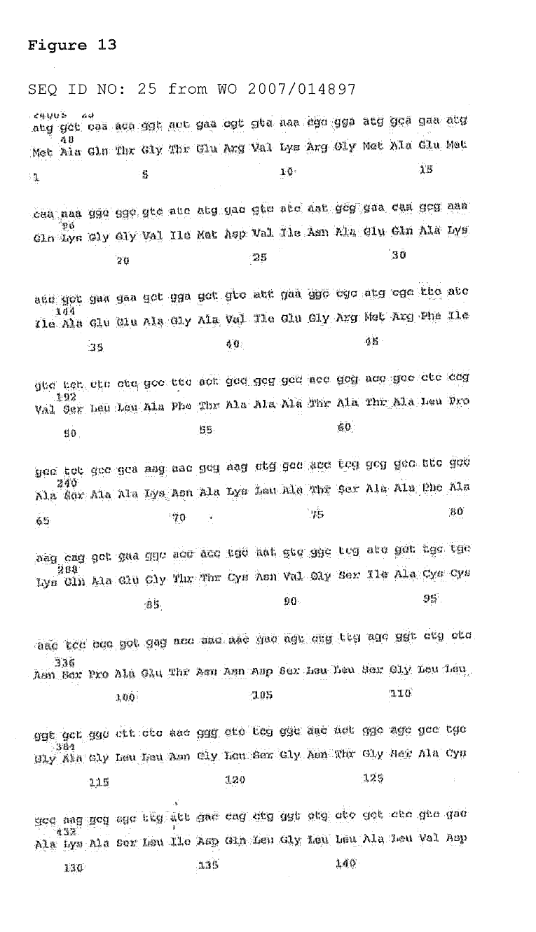

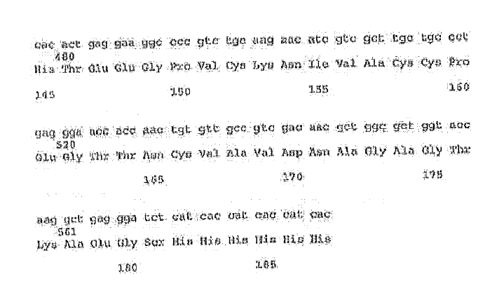

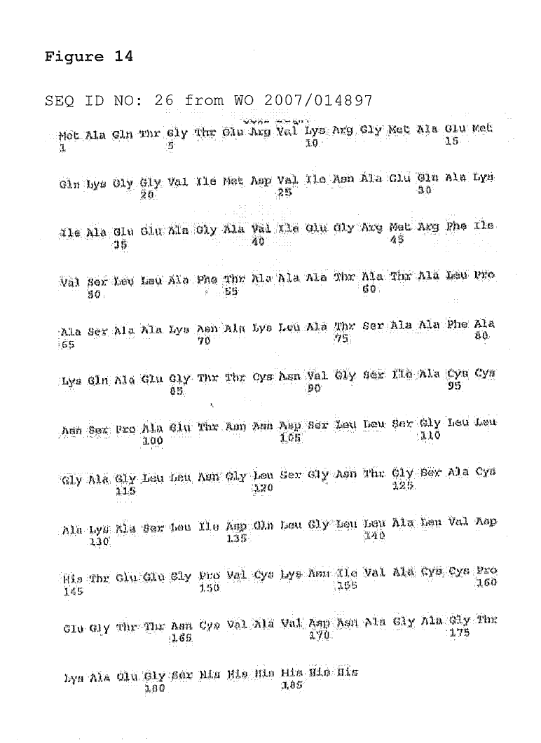

Hydrophobins particularly suitable for carrying out the present invention are furthermore hydrophobins derived from yaad-Xa-dewA-his (SEQ ID NO:20, FIG. 4 of this application), yaad-Xa-rodA-his (SEQ ID NO:22, FIG. 6 of this application) or yaad-Xa-basf1-his (SEQ ID NO:24, FIG. 8 of this application) by shortening the yaad fusion partner. Instead of the complete yaad fusion partner (SEQ ID NO:16, FIG. 1 of this application) with 294 amino acids a shortened yaad radical can be used. The shortened radical, however, should comprise at least 20, preferably at least 35, contiguous amino acids of the yaad sequence. For example, a shortened radical with 20-293, preferably 25 to 250, particularly preferably 35 to 150 and very particularly preferably 35 to 100, amino acids can be used. A particularly suitable protein is yaad40-Xa-dewA-his (SEQ ID NO:25 and 26 in WO 2007/014897, see FIGS. 13 and 14 of this application), which has a yaad radical shortened to 40 amino acids.

A cleavage site between the fusion partner or fusion partners and the radical of the polypeptide can be utilized for cleaving off the fusion partner (for example by BrCN cleavage on methionine, factor Xa, enterokinase, thrombin, TEV cleavage etc.). Particular preference is given to a Xa cleavage site, e.g. a cleavage site of the hydrophobins used in the examples.

As already explained above, hydrophobins are surface-active polypeptides. They can be isolated from natural sources, but can also be obtained by means of genetic engineering methods. In principle, hydrophobins of the one or other origin are suitable for carrying out the present invention.

The hydrophobins used according to the invention can be prepared chemically by known methods of peptide synthesis, for example by solid-phase synthesis in accordance with Merrifield.

Naturally occurring hydrophobins can be isolated from natural sources by means of suitable methods. For example, reference may be made to Wösten et al., Eur. J Cell Bio. 63, 122-129 (1994) or WO 96/41882.

A genetic engineering production method for hydrophobins from Talaromyces thermophilus which comprise no fusion partner is described e.g. in US 2006/0040349.

The preparation of hydrophobins which comprise a fusion partner can preferably take place by genetic engineering methods in which one nucleic acid sequence which codes for the fusion partner and a nucleic acid sequence which codes for the radical of the polypeptide, in particular DNA sequence, are combined such that the desired protein is produced in a host organism as a result of gene expression of the combined nucleic acid sequence. Such a preparation process is disclosed for example by WO 2006/082251 or WO 2006/082253. The fusion partners considerably facilitate the preparation of the hydrophobins. Hydrophobins which comprise a fusion partner are produced in the genetic engineering methods with considerably better yields than hydrophobins which comprise no fusion partner.

The hydrophobins produced by the genetic engineering methods from the host organisms can be worked-up in a manner known in principle and be purified by means of known chromatographic methods.

In general, isolated, in particular purified hydrophobins are used for carrying out the invention.

In a preferred embodiment, the simplified work-up and purification methods disclosed in WO 2006/082253, pages 11/12 can be used.

For this, the fermented cells are firstly separated off from the fermentation broth and disrupted, and the cell debris is separated off from the inclusion bodies. The latter can advantageously take place by centrifugation. Finally, the inclusion bodies can be disrupted for example by acids, bases and/or detergents in a manner known in principle in order to release the hydrophobins. The inclusion bodies with the hydrophobins used according to the invention can generally be completely dissolved within ca. 1 h using just 0.1 M NaOH.

The resulting solutions can, optionally after establishing the desired pH, be used without further purification for carrying out this invention. The hydrophobins can, however, also be isolated from the solutions as solid. Preferably, the isolation can take place by means of spray granulation or spray drying, as described in WO 2006/082253, page 12. The products obtained by the simplified work-up and purification method comprise, besides remains of cell debris, generally ca. 80 to 90% by weight of proteins. The amount of hydrophobins is generally from 30 to 80% by weight, with regard to the amount of all proteins, depending on the fermentation conditions.

The isolated hydrophobin-comprising products can be stored as solids.

The hydrophobins can be used for carrying out this invention as such or else following cleavage and separation of the fusion partner. A cleavage is advantageously carried out after the isolation of the inclusion bodies and their dissolution.

One biological property of the hydrophobins is the change in surface properties when a surface, e.g. a glass surface, is coated with hydrophobin proteins. The change in the surface properties can be determined experimentally, for example by measuring the contact angle of a drop of water before and after coating the surface with the proteins and ascertaining the difference between the two measurements.

The procedure of measuring the contact angles is known in principle to the person skilled in the art. The measurements are carried out for example at room temperature with a water drop of 5 μl and using glass plates as substrate. The precise experimental conditions for a method, suitable by way of example, for measuring the contact angle are explained in example 10 of WO 2006/136607. Under the conditions specified therein, the hydrophobins used can increase the contact angle. For example, the hydrophobins can increase the contact angle for example by at least 20°, preferably at least 25°, particularly preferably at least 30°, at least 40°, at least 45°, in particular at least 50°, in each case compared with the contact angle of an identically sized water drop with the uncoated glass surface.

Within the context of the present invention, hydrophobin is used as a spreading agent. With regard to the contact angle, this means that the hydrophobin increases the contact angle of a hydrophobin-containing composition according to the invention with a surface, for example by at least 5%, preferably at least 20°, particularly preferably at least 25°, at least 30°; at least 40°; at least 45°, in particular at least 50°, in each case compared with the contact angle of an identically sized drop of the composition without hydrophobin with the untreated surface. Alternatively, the hydrophobin as a spreading agent increases the contact angle to the same extent when a surface is treated firstly with hydrophobin and then with a further composition.

The present invention relates to the effect of hydrophobin on the spreading behavior of compositions, in particular of cosmetic or pharmaceutical compositions. The binding of hydrophobin to skin and hair can be tested as described in examples 1 to 4 (identical to examples 11 to 14 in WO 2006/136607). The spreading behavior can be tested as described in example 5.

The composition according to the invention as in embodiment (4), and the compositions arising as a result of using hydrophobin as in embodiment (1) to (3) comprise the hydrophobin preferably in a concentration of from 0.001 to 10% by weight, preferably from 0.01 to 5% by weight, particularly preferably an amount from 0.05 to 3% by weight, very particularly preferably from 0.1 to 1% by weight, of total hydrophobin, based on the total composition. A hydrophobin concentration of up to 0.2% by weight is most preferred, in particular of from 0.01 to 0.05% by weight, primarily of 0.025% by weight. The total hydrophobin is the total amount of the hydrophobin molecules of one or more types of hydrophobin molecule in the composition.

These concentration ranges also indicate the preferred concentrations in which the hydrophobin is applied to the treated surface when used in accordance with embodiment (1) to (3).

The specified hydrophobin concentrations are present for example either already in the compositions according to the invention as in embodiment (4), or the compositions used when using the hydrophobin according to embodiment (1) to (3), or they are obtained by diluting a concentrate before using the composition.

The composition according to the invention can comprise only one hydrophobin or else a combination of different hydrophobins, e.g. a composition which comprises two or three hydrophobins. Equally, according to embodiment (1) to (3), one hydrophobin or a combination of different hydrophobins can be used.

The substances present in a composition according to the invention besides hydrophobin can in principle be hydrophobic or hydrophilic. In a preferred aspect of the present invention, at least one further ingredient besides the hydrophobin and the carrier medium in the embodiments (1) to (4) is a hydrophilic ingredient, in particular a hydrophilic active ingredient or a hydrophilic effector molecule. The hydrophobin improves in particular its binding to the surface of the treated tissue, such as e.g. the skin, since it can make the surface more hydrophilic.

On the other hand, hydrophobic substances are able to penetrate a hydrophobin layer on the surface and accumulate in the deeper layers. Hydrophobic substances stabilized in this way remain longer on the treated tissue. Their use as ingredients in a hydrophobin-containing composition according to the invention is therefore likewise an aspect of the present invention.

The use of at least one hydrophilic active ingredient is preferred in all uses and compositions according to the invention. Particularly preferably, the greater part of the active ingredients present is hydrophilic (more than 50% by weight, based on the total active ingredients), and very particularly preferably all of the active ingredients present are hydrophilic. The same applies to effector molecules.

Finally, hydrophobin can also improve the absorption and residence time of active ingredients which are applied to the surface at the same time as or after the hydrophobin. Hydrophobin can serve in particular as penetration promoter, i.e. for increasing the amount of a further substance, in particular of an active ingredient, which penetrates into the surface or passes through the surface per unit of time.

Hydrophobin as a spreading agent can furthermore change the surface properties of the treated surface such that the spreading behavior of compositions applied subsequently is changed. This applies in particular when pretreating the surface to be treated with a hydrophobin-comprising composition before applying a further composition, the spreading of which on the surface should be promoted by the hydrophobin as a spreading agent.

The use according to embodiment (1) to (3) can take place in the course of a process which comprises one or more steps. Preferably, the process comprises at least the following steps:
(a) applying at least one hydrophobin, e.g. a hydrophobin of the structural formula (I), to the surface to be treated; and
(b) applying at least one further, usually non-hydrophobin-comprising composition to the hydrophobin-treated surface, where steps (a) and (b) are carried out either simultaneously or in succession.

If steps (a) and (b) are carried out simultaneously, then only one composition is applied which then comprises both the hydrophobin and also the ingredients of the further composition. This only one composition can be prepared by mixing two separate compositions, of which the one comprises the hydrophobin.

If steps (a) and (b) are carried out in succession (i.e. step (a) before step (b)), then the hydrophobin, preferably in the form of a hydrophobin-containing composition, is firstly applied to the treated surface, e.g. the skin. This composition acts on the surface for a certain contact time. Then, a further composition is applied to the surface. The application of the last-mentioned composition can follow directly after the contact time of the hydrophobin-containing composition, or yet further steps (i.e. one or more steps) are carried out between step (a) and (b).

These further steps may all be measures which serve for surface treatment provided they neither remove the hydrophobin layer nor adversely affect the end result of the surface treatment. Such a step is preferably the drying of the surface after applying the hydrophobin-containing composition.

The steps of the process can also be repeated several times.

The sequence and nature of the individual steps in example 5 is one preferred way of carrying out the process (1) according to the invention. A selection can also be made from these steps provided this corresponds to the provisos specified above.

In one preferred aspect of the present invention, the hydrophobin is used as a spreading agent in a pharmaceutical or cosmetic composition. Preference is given to cosmetic compositions.

As regards the ingredients which can be used according to the invention in pharmaceutical and cosmetic compositions besides the hydrophobin, reference is expressly made to the online database “CosIng” of the European Union (http://ec.europa.eu/enterprise/cosmetics/cosing/), which comprises the “European Inventory of Cosmetic Raw Materials”, published by the European Community, and within this database in particular to substances with the function “skin conditioning”, “skin protecting” and/or “smoothing”, and also to the “European Inventory of Cosmetic Raw Materials” itself, published by the European Community, available from the Bundesverband Deutscher Industrie—und Handelsunternehmen für Arzneimittel, Reformwaren and Körperpflegemittel e.V., Mannheim.

Besides the cosmetic preparations, there is a large number of medicinal topical compositions which as a rule comprise one or more pharmaceutically active substances in an effective concentration. These compositions too are in need of improvement both with regard to their spreadability on the skin and also their sensory properties. For distinguishing between cosmetic and medicinal application and corresponding products, reference is made to the legal provisions of the Federal Republic of Germany (e.g. Cosmetics Ordinance, Food and Drugs Act).

In a preferred aspect of the embodiments (1) to (4), the hydrophobin is a constituent of a composition for treating external body surfaces such as the skin, the hair or the nails, in particular for treating human hair, skin or nails. The composition is particularly preferably a cream, an ointment, a lotion or an aqueous solution such as e.g. a spray. It is very particularly preferably a composition for treating large skin areas, such as a sunscreen composition (spray, lotion or cream), a skin care product, or an insect protection composition. Or it is a pigment- or dye-containing composition, the uniform distribution of which is desirable for avoiding uneven colorations of the skin or of the hair, such as a self-tanning composition, a foundation, a make-up or a hair colorant. Particularly in the case of such preparations, a good spreading behavior is desired for facilitating simple and uniform distribution of the preparation over the entire large skin area.

Besides the hydrophobin, a cosmetic or pharmaceutical composition according to the invention generally comprises at least one cosmetically or pharmaceutically acceptable carrier medium and can in addition also comprise further ingredients customary in cosmetics or pharmaceuticals.

It has been found that the use of hydrophobin and spreading agent can have further advantageous effects besides the aforementioned advantages. For example, the application of hydrophobin increases the intensity and resistance of the coloration by hair colorants (cf. EP 08 16 2556.8) and can also extend the residence time of other cosmetic ingredients on hair (US 2003/0217419 A1).

Besides hydrophobin, the cosmetic or pharmaceutical compositions according to the invention generally comprise a cosmetically and/or pharmaceutically acceptable medium and also suitable further ingredients which assist the cosmetic and/or pharmaceutical effect, in particular auxiliaries and additives customary in cosmetics and pharmaceuticals. These constituents should not adversely affect the treatment result. Base formulations of this type for cosmetic and pharmaceutical compositions and the ingredients suitable therefor are sufficiently known to the person skilled in the art and can be found in cosmetics handbooks, such as e.g. in Schrader, Grundlagen und Rezepturen der Kosmetika [Fundamentals and Formulations of Cosmetics], Hüthig Verlag, Heidelberg, 1989, or Umbach, Kosmetik: Entwicklung, Herstellung und Anwendung kosmetischer Mittel [Cosmetics: Development, Preparation and Use of Cosmetic Compositions], second expanded edition, 1995, Georg Thieme Verlag, or Bauer, Frömming, Führer, Lehrbuch der pharmazeutischen Technologie [Textbook of Pharmaceutical Technology], 7th edition, Wissenschaftliche Verlagsgesellschaft mbH Stuttgart, 2002.

The compositions according to the invention are formulated inter alia as cosmetic or pharmaceutical preparations, for example as creams, emulsions, gels or else surfactant-containing foaming solutions, e.g. shampoos, foam aerosols or other preparations which are suitable for application to body surfaces, in particular to skin and/or hair.

Customary constituents of such water-containing cosmetic and/or pharmaceutical preparations are e.g. wetting agents and emulsifiers, such as anionic, nonionic and ampholytic surfactants, e.g. fatty alcohol sulfates, alkanesulfonates, α-olefinsulfonates, fatty alcohol polyglycol ether sulfates, ethylene oxide addition products onto fatty alcohols, fatty acids and alkylphenols, sorbitan fatty acid esters and fatty acid partial glycerides, fatty acid alkanolamides, and thickeners, such as e.g. methyl- and hydroxyethyl-cellulose, starch, fatty alcohols, paraffin oils, fatty acids, also perfume oils and skin and/or hair care additives, such as e.g. water-soluble cationic ampholytic and anionic polymers, protein derivatives, pantothenic acid, cholesterol, dyes, active ingredients such as panthenol, allantoin, pyrrolidonecarboxylic acids and salts thereof, plant extracts and vitamins, photoprotective agents, consistency regulators such as sugar esters, polyol esters or polyol alkyl ethers, waxes, such as beeswax and montan wax, complexing agents such as EDTA, NTA and phosphonic acids, swelling and penetration substances such as glycerol, propylene glycol monoethyl ether, carbonates, hydrogencarbonates, guanidines, ureas, and primary, secondary and tertiary phosphates, pearlizing agents such as ethylene glycol mono- and distearate, propellants such as propane/butane mixtures, N₂O, dimethyl ether, CO₂ and air, and also antioxidants.

Further customary constituents and the preparation of water-containing cosmetics and pharmaceuticals are known to the person skilled in the art and are described for example in Schrader. Grundlagen and Rezepturen der Kosmetika [Fundamentals and formulations of cosmetics], Hüthig Buch Verlag, Heidelberg, 2nd edition, 1989, and also in WO 2007/063024 and in WO 2006/136607 as cosmetically suitable auxiliaries and additives. Reference is expressly made thereto. The constituents of the cosmetic carriers are used for producing the compositions according to the invention in amounts customary for these purposes. However, the content of oil components and/or emulsifiers is preferably reduced compared with conventional pharmaceutical and cosmetic compositions; oil bodies and emulsifiers are particularly preferably missing entirely in the compositions according to the invention.

As a rule, the cosmetic or pharmaceutical preparation is used for application to the skin (topical), teeth or hair. Topical preparations are to be understood here as meaning those preparations which are suitable for applying one or more active ingredients to the skin in fine distribution and preferably in a form which can be absorbed by the skin. Of suitability for this purpose are e.g. according to the invention aqueous and aqueous-alcoholic solutions, sprays, foams, foam aerosols, ointments, aqueous gels, emulsions of the O/W or W/O type, microemulsions or cosmetic stick preparations. Preparations which include no or only a small amount of (preferably less than 20% by weight, particularly preferably less than 10% by weight, very particularly preferably less than 5% by weight) of oil bodies are preferred.

The cosmetic or pharmaceutical composition according to the invention for surface treatment comprises a carrier. A preferred carrier is water, a water-based or hydrophilic liquid, a gel, an ointment, a cream, an emulsion or microemulsion, a dispersion or a mixture thereof. The specified carriers exhibit good skin compatibility. Hydrophilic gels, hydrophilic ointments or hydrophilic creams are particularly advantageous.

Irrespective of the type of cosmetic or pharmaceutical preparation, e.g. as cream, gel or shampoo, the compositions according to the invention can have a weakly acidic, neutral or alkaline pH. Preference is given to a pH range from 6 to 8. The pH is adjusted with the help of customary pH extenders, but preferably not with ammonia or other chemicals which are regarded as harmful.

In a preferred aspect, besides the hydrophobin, additionally at least one cosmetically or pharmaceutically active ingredient, the absorption and/or effect of which is improved through the presence of hydrophobin, is applied to the treated surface. In this preferred aspect, either the compositions according to the invention additionally comprise, besides hydrophobin, at least one cosmetically or pharmaceutically active ingredient, the absorption and/or effect of which is improved through the presence of hydrophobin, or this cosmetically or pharmaceutically active ingredient is applied separately (in pure form or as a constituent of a composition).

The particular quantitative and qualitative effects are determined by the individual cosmetic or pharmaceutical active ingredients and their field of application. In particular, further constituents of creams, e.g. care substances, can have a more intensive effect as a result of the presence of hydrophobin.

The additional cosmetically or pharmaceutically active ingredient is preferably hydrophilic.

The use according to the invention of hydrophobin as a spreading agent is preferably not a therapeutic treatment of the human or animal body. In this respect, it serves merely for nontherapeutic purposes, such as improving the distribution of the hydrophobin-containing composition on the treated surface. In connection with the treatment of human skin and other human body surfaces, it serves in particular for cosmetic purposes, thus is preferably a cosmetic use.

Preferred cosmetically active ingredients, the absorption of which is improved by hydrophobin, are described in WO 2006/136607 as “effector molecules”, to the corresponding passages of which reference is hereby expressly made.

In the compositions according to the invention, in one embodiment of the present invention, effector molecules can be used as cosmetically active ingredients.

Hereinbelow, effector molecules are understood as meaning molecules which have a certain, predictable effect. These may be either protein-like molecules, such as enzymes, or else non-proteinogenic molecules such as dyes, photoprotective agents, vitamins and fatty acids, sugars or compounds containing metal ions.

Among the sugars, glucans and in particular sugars of natural origin, such as e.g. from honey or cereals, are preferred.

Among the protein-like effector molecules, enzymes, peptides and antibodies are preferred.

Among the enzymes, the following are preferred as effector molecules: oxidases, peroxidases, proteases, tyrosinases, metal-binding enzymes, lactoperoxidase, lysozyme, amyloglycosidase, glucose oxidase, superoxide dismutase, photolyase, catalase.

Highly suitable protein-like effector molecules are also hydrolysates of proteins from vegetable and animal sources, for example hydrolysates of proteins of marine origin, milk hydrolysates or soap hydrolyates.

Of particularly good suitability are defined peptides which are used for antiaging, such as Matrixyl (INCI name Glycerin-Water-Butylene Glycol Carbomer-Polysorbate 20-Palmitoyl Pentapeptide-4), Argireline (INCI name Aqua, AcetyHexapeptide-3), Rigin (INCI name Water (and)—Glycerin (and) Steareth-20 (and) Palmitoyltetrapeptide-7), Eyeliss (INCI name Water-Glyerin-Hespiridin Methyl Chalcone-Steareth-20-Dipeptide-2-Palmitoyl Tetrapeptide-7), Regu-Age (INCI name Oxido Reductases-Soy Peptides-Hydrolyzed Rice Bran Extract) and Melanostatin-5 (INCI name Aqua-dextran-Nonapetide-1).

Among the non-protein-like effector molecules, preference is given to non-protein anti-aging agents such as e.g. caffeine, and antioxidants as effector molecules. Antioxidants, which are also referred to as radical scavengers, are able to neutralize so-called free radicals. These are aggressive compounds which are formed physiologically in numerous metabolic processes and the generation of energy. They are important for defence reactions by the body, but can also bring about damage to genetic material (DNA), the cell membranes and body proteins. This damage can lead to premature tissue aging, tissue death and cancer. The antioxidants include carotenoids, ascorbic acid (vitamin C, E 300) and also sodium L-ascorbate (E 301) and calcium L-ascorbate (E 302); ascorbyl palmitate (E E304); butylhydroxyanisole (E 320); butylhydroxytoluene (E 321); calcium-disodium-EDTA (E 385); gallate and also propyl gallate (E 310), octyl gallate (E 311) and dodecyl gallate (lauryl gallate)(E 312); isoascorbic acid (E 315) and also sodium isoascorbate (E 316); lecithin (E 322); lactic acid (E 270); multiphosphates such as diphosphates (E 450), triphosphates (E 451) and polyphosphates (E 452); sulfur dioxide (E 220) and also sodium sulfite (E 221), sodium bisulfite (E 222), sodium disulfite (E 223), potassium sulfite (E 224), calcium sulfite (E 226), calcium hydrogensulfite (E 227) and potassium bisulfite (E 228); selenium; tocopherol (vitamin E, E 306) and also alpha-tocopherol (E 307), gamma-tocopherol (E 308) and delta-tocopherol (E 309); tin II chloride (E 512); citric acid (E 330) and also sodium citrate (E 331) and potassium citrate (E 332); L-glutathione, L-cysteine, polyphenols, phenolic acids, flavonoids, phytoestrogens, glutathione and the antioxidative enzymes superoxide dismutase, glutathione peroxidas and catalase. According to the invention, at least one compound from the group of antioxidants specified above is selected as antioxidant.

Further suitable effector molecules are carotenoids. According to the invention, carotenoids are to be understood as meaning the following compounds: beta-carotene, lycopene, lutein, astaxanthin, zeaxanthin, cryptoxanthin, citranaxanthin, canthaxanthin, bixin, beta-apo-4-carotenal, beta-apo-8-carotenal, beta-apo-8-carotenoic acid ester, individually or as a mixture. Preferably used carotenoids are beta-carotene, lycopene, lutein, astaxanthin, zeaxanthin, citranaxanthin and canthaxanthin.

Within the context of the present invention, retinoids are to be understood as meaning vitamin A alcohol (retinol) and its derivatives, such as vitamin A aldehyde (retinal), vitamin A acid (retinoic acid) and vitamin A ester (e.g. retinyl acetate, retinyl propionate and retinyl palmitate). The term retinoic acid here comprises both all-trans retinoic acid and also 13-cis retinoic acid. The terms retinol and retinal preferably comprise the all-trans compounds. The preferred retinoid used is all-trans retinol, referred to below as retinol.

Further preferred effector molecules are vitamins, in particular vitamin A, and esters thereof.

Vitamins are essential organic compounds which are either not synthesized or are synthesized only in inadequate amounts in the animal and human organism. On the basis of this definition, 13 components or groups of components have been classified as vitamins. The fat-soluble vitamins include vitamin A (retinols), vitamin D (calciferols), vitamin E (tocopherols, tocotrienols) and vitamin K (phylloquinones). The water-soluble vitamins include vitamin B1 (thiamine), vitamin B2 (riboflavin), vitamin B6 (pyridoxal group). vitamin B12 (cobalamine), vitamin C (L-ascorbic acid), pantothenic acid, biotin, folic acid and niacin.

Vitamins, provitamins and vitamin precursors from the groups A, C, E and F, in particular 3,4-didehydroretinol, beta-carotene (provitamin of vitamin A), ascorbic acid (vitamin C), and the palmitic acid esters, glucosides or phosphates of ascorbic acid, tocopherols, in particular a-tocopherol, and its esters, e.g. the acetate, the nicotinate, the phosphate and the succinate; also vitamin F, which is understood as meaning essential fatty acids, particularly linoleic acid, linolenic acid and arachidonic acid.

Vitamin E is a collective term for a group of (to date) eight fat-soluble substances with antioxidative and non-antioxidative effects. Vitamin E is a constituent of all membranes of animal cells, but is formed only by photosynthetically active organisms such as plants and cyanobacteria. Four of the eight known vitamin E forms are called tocopherols (alpha-tocopheraol, beta-tocopherol, gamma-tocopherol and dela-tocopherol). The other hitherto known four forms of vitamin E are called tocotrienols (alpha-tocotrienol, beta-tocotrienol, gamma-tocotrienol and delta-tocotrienol). In addition, derivatives of these substances, such as alpha-tocopheryl acetate, may also be advantageous.

Vitamin A and its derivatives and provitamins advantageously exhibit a particular skin smoothing effect.

The vitamins, provitamins or vitamin precursors of the vitamin B group or derivatives thereof and also the derivatives of 2-furanone that are preferably to be used according to the invention include inter alia:
Vitamin B1, trivial name thiamine, chemical name 3[(4′-amino-2′-methyl-5′-pyrimidinyl)methyl]-5-(2-hydroxyethyl)-4-methylthiazolium chloride.
Vitamin B2, trivial name riboflavin, chemical name 7,8-dimethyl-10-(1-D-ribityl)-benzo[g]pteridine-2,4(3H,10H)-dione. In free form, riboflavin occurs e.g. in whey, other riboflavin derivatives can be isolated from bacteria and yeasts. A stereoisomer of riboflavin which is likewise suitable according to the invention is lyxoflavin, which can be isolated from fish meal or liver and carries a D-arabityl radical instead of the D-ribityl radical.
Vitamin B3. This name is often used for the compounds nicotinic acid and nicotinamide (niacinamide). According to the invention, preference is given to nicotinamide.
Vitamin B5 (pantothenic acid and panthenol). Preference is given to using panthenol. Derivatives of panthenol which can be used according to the invention are in particular the esters and ethers of panthenol, and cationically derivatized panthenols. In a further preferred embodiment of the invention, derivatives of 2-furanone can also be used in addition to pantothenic acid or panthenol. Particularly preferred derivatives are the also commercially available substances dihydro-3-hydroxy-4,4-dimethyl-2(3H)-furanone with the trivial name pantolactone (Merck), 4-hydroxymethyl-γ-butyrolactone (Merck), 3,3-dimethyl-2-hydroxy-γ-butyrolactone (Aldrich) and 2,5-dihydro-5-methoxy-2-furanone (Merck), with all stereoisomers being expressly included.
   These compounds advantageously impart moisturizing and skin calming properties to the compositions according to the invention.
Vitamin B6, which is understood as meaning not a uniform substance, but the derivatives of 5-hydroyxymethyl-2-methylpyridin-3-ol known under the trivial names pyridoxine, pyridoxamine and pyridoxal.
Vitamin B7 (biotin), also referred to as vitamin H or “skin vitamin”. Biotin is (3aS,4S, 6aR)-2-oxo-hexahydrothienol[3,4-d]imidazole-4-valeric acid.
Vitamin B9 and vitamin B12.

According to the invention, suitable derivatives of vitamins (salts, esters, sugars, nucleotides, nucleosides, peptides and lipids) can likewise be used.

Nucleic acids, such as DNA and RNA, can also be suitable effector molecules, e.g. as moisture donors.

As lipophilic, oil-soluble antioxidants from this group, preference is given to tocopherol and derivatives thereof, gallic acid esters, flavonoids and carotenoids, and also butylhydroxytoluene/anisole. Preferred water-soluble antioxidants are amino acids, e.g. tyrosine and cysteine and derivatives thereof, and also tannins, in particular those of vegetable origin.

Triterpenes, in particular triterpenoic acids, such as ursolic acid, rosmaric acid, betulinic acid, boswellic acid and bryonolic acid are likewise preferred.

Further preferred effector molecules are preferably low-dose fruit acids (alpha-hydroxy acids), such as, for example, malic acid, citric acid, lactic acid, tartaric acid, glycolic acid. These may be present in concentrations of from 0.1% to 35%, preferably 0.1% to 10%, in particular 1% to 10%, 1% to 5%, based on the total weight of the composition.

Further preferred effector molecules are urea and derivatives thereof since these can care for the skin. These are present in concentrations of from 0.1% to 25%, preferably 0.1% to 10%, in particular 1% to 10%, 1% to 5%, based on the total weight of the composition.

Further preferred effector molecules are UV photo-protective filters, in particular the UV filters specified in WO 2006/136607.

Particularly preferred further cosmetically active ingredients of this type are keratin care substances and skin care substances, in particular water-soluble vitamins, antioxidants, UV filters, glucans, flavonoids, and caffeine.

From the group of the water-soluble vitamins, preference is in turn given to one or more of the vitamins from the group consisting of vitamin C, vitamin B1, vitamin B2, niacin (nicotinic acid, nicotinamide, vitamin B3), pantothenic acid, pantolactone and panthenol (vitamin B5), vitamin B6, biotin (vitamin B7, vitamin H), vitamin B9 (folic acid) and vitamin B12 or derivatives thereof. Panthenol, pantolactone, nicotinamide, sodium ascorbyl phosphate, and also biotin are very particularly preferred according to the invention.

From the group of UV filters, preference is in turn given to water-soluble UV filters, particularly preferably Uvinul MS 40, Uvinul P 25, Uvinul DS 49 and Z-COTE, and very particularly preferably Uvinul MS 40 and P 25.

From the group of antioxidants, preference is in turn given to flavonoids, phenolic acids and polyphenols.

One or more of the cosmetically active ingredients are most preferably selected from the group consisting of panthenol, ascorbic acid and derivatives thereof, water-soluble UV filters, caffeine.

Aqueous extracts from fruits and herbs, i.e. in particular plant extracts, fruit extracts or herb extracts, such as e.g. from grapes, lime, grapefruit, oats, wheat, rice, soya, gingseng, peppermint etc., may likewise be a constituent of the compositions according to the invention.

The particular quantitative and qualitative effects are determined by the individual cosmetic or pharmaceutical active ingredients and their field of application. In particular, further constituents of creams, e.g. care substances, can have a more intensive effect as a result of the presence of hydrophobin.

In a further preferred aspect, the composition according to the invention for surface treatment is a crop protection composition. The present invention further provides a method for producing crop protection compositions comprising hydrophobin as a spreading agent. In this aspect of the invention, besides the hydrophobin, additionally at least one crop protection active ingredient, the absorption and/or effect of which is improved by the presence of hydrophobin, is applied to the treated surface. In this preferred aspect, either the compositions according to the invention comprise, as well as hydrophobin, additionally at least one crop-protecting ingredient, the absorption and/or effect of which is preferably improved by the presence of hydrophobin, or this ingredient is applied separately (in pure form or as constituent of a composition).

The additional crop-protecting active ingredient is preferably hydrophilic.

One particular embodiment of the invention relates to the formulation of pesticides for crop protection, in particular of herbicides, fungicides, nematicides, acaricides, insecticides, and also active ingredients which regulate plant growth. The term pesticide refers here to at least one active ingredient selected from the group of fungicides, insecticides, nematicides, herbicides, acaricides and/or safeners or growth regulators. Mixtures of pesticides from one, two or more of the aforementioned classes can also be used. The person skilled in the art is familiar with such pesticides, which can be found for example in Pesticide Manual, 13th ed. (2003), The British Crop Protection Council, London.

Examples of fungicidal active ingredients which can be formulated as active ingredient composition according to the invention include:
acylalanines such as benalaxyl, metalaxyl, ofurace, oxadixyl;
amine derivates such as aldimorph, dodine, dodemorph, fenpropimorph, fenpropidin, guazatine, iminoctadine, spiroxamin, tridemorph;
anilinopyrimidines such as pyrimethanil, mepanipyrim or cyrodinyl;
antibiotics such as cycloheximide, griseofulvin, casugamycin, natamycin, polyoxin and streptomycin;
azoles such as bitertanol, bromoconazole, cyproconazole, difenoconazole, dinitroconazole, epoxiconazole, fenbuconazole, fluquiconazole, flusilazole, flutriafol, hexaconazole, imazalil, ipconazole, metconazole, myclobutanil, penconazole, propiconazole, prochloraz, prothioconazole, tebuconazole, tetraconazole, triadimefon, triadimenol, triflumizole, triticonazole;
2-methoxybenzophenones, as are described in EP-A 897904 by the general formula I, e.g. metrafenone;
dicarboximides such as iprodione, myclozolin, procymidone, vinclozolin;
dithiocarbamates such as ferbam, nabam, maneb, mancozeb, metam, metiram, propineb, polycarbamate, thiram, ziram, zineb;
heterocyclic compounds such as anilazine, benomyl, boscalid, carbendazim, carboxin, oxycarboxin, cyazofamid, dazomet, dithianon, famoxadone, fenamidone, fenarimol, fuberidazole, flutolanil, furametpyr, isoprothiolane, mepronil, nuarimol, picobezamid, probenazole, proquinazid, pyrifenox, pyroquilon, quinoxyfen, silthiofam; thiabendazole, thifluzamid, thiophanate-methyl, tiadinil, tricyclazole, triforine;
nitrophenyl derivatives such as binapacryl, dinocap, dinobuton, nitrophthal-isopropyl;
phenylpyrroles such as fenpiclonil and fludioxonil;
unclassified fungicides such as acibenzolar-S-methyl, benthiavalicarb, carpropamid, chlorothalonil, cyflufenamid, cymoxanil, diclomezin, diclocymet, diethofencarb, edifenphos, ethaboxam, fenhexamid, fentin acetate, fenoxanil, ferimzone, fluazinam, fosetyl, fosetyl aluminum, iprovalicarb, hexachlorobenzene, metrafenone, pencycuron, propamocarb, phthalide, toloclofos-methyl, quintozene, zoxamide;
strobilurins as described in WO 03/075663 by the general formula I, for example azoxystrobin, dimoxystrobin, fluoxastrobin, kresoxim-methyl, metominostrobin, orysastrobin, picoxystrobin, pyraclostrobin and trifloxystrobin;
sulfenic acid derivatives such as captafol, captan, dichlofluanid, folpet, tolylfluanid;
cinnamides and analogues such as dimethomorph, flumetover, flumorp;
6-aryl-[1,2,4]triazolo[1,5-a]pyrimidines as described e.g. in WO 98/46608, WO 99/41255 or WO 03/004465 in each case by the general formula I (page 1, line 8 to page 11, line 45, and also compounds depicted in formula IA in conjunction with tables 1 to 44 and table A in WO 03/00465);
amide fungicides such as cyclofenamid and also (Z)-N-[α-(cyclopropylmethoxyimino)-2,3-difluoro-6-(difluoromethoxy)benzyl]-2-phenylacetamide.

Examples of herbicides which can be formulated as active ingredient composition according to the invention include:
1,3,4-thiadiazoles such as buthidazole and cyprazole;
amides such as allidochlor, benzoylpropethyl, bromobutide, chlorthiamid, dimepiperate, dimethenamid, diphenamid, etobenzanid, flampropmethyl, fosamin, isoxaben, metazachlor, alachlor, acetochlor, metolachlor, monalide, naptalam, pronamid, propanil;
aminophosphoric acids such as bilanafos, buminafos, glufosinate ammonium, glyphosate, sulfosate;
aminotriazoles such as amitrol;
anilides such as anilofos, mefenacet;
aryloxyalkanoic acids such as 2,4-D, 2,4-DB, clomeprop, dichlorprop, dichlorprop-P, fenoprop, fluroxypyr, MCPA, MCPB, mecoprop, mecoprop-P, napropamide, napropanilide, triclopyr;
benzoic acids such as chloramben, dicamba;
benzothiadiazinones such as bentazone;
bleachers such as clomazone, diflufenican, fluorochloridone, flupoxam, fluridone, pyrazolate, sulcotrione;
carbamates such as carbetamid, chlorbufam, chlorpropham, desmedipham, phenmedipham, vernolate;
quinolinic acids such as quinclorac, quinmerac;
dichloropropionic acids such as dalapon;
dihydrobenzofurans such as ethofumesate;
dihydrofuran-3-ones such as flurtamone;
dinitroanilines such as benefin, butralin, dinitramine, ethalfluralin, fluchloralin, isopropalin, nitralin, oryzalin, pendimethalin, prodiamine, profluralin, trifluralin, dinitrophenols such as bromofenoxim, dinoseb, dinoseb acetate, dinoterb, DNOC, minoterb acetate;
diphenyl ethers such as acifluorfen-sodium, aclonifen, bifenox, chlornitrofen, difenoxuron, ethoxyfen, fluorodifen, fluoroglycofen-ethyl, fomesafen, furyloxyfen, lactofen, nitrofen, nitrofluorfen, oxyfluorfen;
dipyridyls such as cyperquat, difenzoquat methyl sulfate, diquat, paraquat dichloride;
imidazoles such as isocarbamid;
imidazolinones such as imazamethapyr, imazapyr, imazaquin, imazethabenz-methyl, imazethapyr, imazapic, imazamox;
oxadiazoles such as methazole, oxadiargyl, oxadiazon;
oxiranes such as tridiphane;
phenols such as bromoxynil, ioxynil;
phenoxyphenoxypropionic acid esters such as clodinafop, cyhalofop-butyl, diclofop-methyl, fenoxaprop-ethyl, fenoxaprop-p-ethyl, fenthiapropethyl, fluazifop-butyl, fluazifop-p-butyl, haloxyfop-ethoxyethyl, haloxyfop-methyl, haloxyfop-p-methyl, isoxapyrifop, propaquizafop, quizalofop-ethyl, quizalofop-p-ethyl, quizalofop-tefuryl;
phenylacetic acids such as chlorfenac;
phenylpropionic acids such as chlorophenprop-methyl;
ppi active ingredients such as benzofenap, cinidon-ethyl, flumiclorac-pentyl, flumioxazin, flumipropyn, flupropacil, pyrazoxyfen, sulfentrazone, thidiazimin;
pyrazoles such as nipyraclofen;
pyridazines such as chloridazon, maleic hydrazide, norflurazon, pyridate;
pyridinecarboxylic acids such as clopyralid, dithiopyr, picloram, thiazopyr;
pyrimidyl ethers such as pyrithiobac acid, pyrithiobac-sodium, KIH-2023, KIH-6127;
sulfonamides such as flumetsulam, metosulam;
triazolecarboxamides such as triazofenamid;
uracils such as bromacil, lenacil, terbacil;
also benazolin, benfuresate, bensulide, benzofluor, bentazon, butamifos, cafenstrole, chlorthal-dimethyl, cinmethylin, dichlobenil, endothall, fluorbentranil, mefluidide, perfluidone, piperophos, topramezone and prohexanedione-calcium;
sulfonylureas such as amidosulfuron, azimsulfuron, bensulfuron-methyl, chlorimuron-ethyl, chlorsulfuron, cinosulfuron, cyclosulfamuron, ethametsulfuron-methyl, flazasulfuron, halosulfuron-methyl, imazosulfuron, metsulfuron-methyl, nicosulfuron, primisulfuron, prosulfuron, pyrazosulfuron-ethyl, rimsulfuron, sulfometuron-methyl, thifensulfuron-methyl, triasulfuron, tribenuron-methyl, triflusulfuron-methyl, tritosulfuron;
crop protection active ingredients of the cyclohexenone type such as alloxydim, clethodim, cloproxydim, cycloxydim, sethoxydim and tralkoxydim. Very particularly preferred herbicidal active ingredients of the cyclohexenone type are: tepraloxydim (cf. AGROW, No. 243, 11.3.95, page 21, caloxydim) and 2-(1-[2-{4-chlorophenoxy}propyloxyimino]butyl)-3-hydroxy-5-(2H-tetrahydrothiopyran-3-yl)-2-cyclohexen-1-one and of the sulfonylurea type: N-(((4-methoxy-6-[trifluoromethyl]-1,3,5-triazin-2-yl)amino)carbonyl)-2-(trifluoromethyl)benzenesulfonamide.

Examples of insecticides which can be formulated as active ingredient composition according to the invention comprise:
organophosphates such as acephate, azinphos-methyl, chlorpyrifos, chlorfenvinphos, diazinon, dichlorvos, dimethylvinphos, dioxabenzofos, dicrotophos, dimethoate, disulfoton, ethion, EPN, fenitrothion, fenthion, isoxathion, malathion, methamidophos, methidathion, methyl-parathion, mevinphos, monocrotophos, oxydemeton-methyl, paraoxon, parathion, phenthoate, phosalone, phosmet, phosphamidon, phorate, phoxim, pirimiphos-methyl, profenofos, prothiofos, primiphos-ethyl, pyraclofos, pyridaphenthion, sulprophos, triazophos, trichlorfon; tetrachlorvinphos, vamidothion
carbamates such as alanycarb, benfuracarb, bendiocarb, carbaryl, carbofuran, carbosulfan, fenoxycarb, furathiocarb, indoxacarb, methiocarb, methomyl, oxamyl, pirimicarb, propoxur, thiodicarb, triazamate;
pyrethroids such as bifenthrin, cyfluthrin, cycloprothrin, cypermethrin, deltamethrin, esfenvalerate, ethofenprox, fenpropathrin, fenvalerate, cyhalothrin, lambda-cyhalothrin, permethrin, silafluofen, tau-fluvalinate, tefluthrin, tralomethrin, alpha-cypermethrin, zeta-cypermethrin, permethrin;
arthropodal growth regulators: a) chitin synthesis inhibitors, e.g. benzoylureas such as chlorfluazuron, diflubenzuron, flucycloxuron, flufenoxuron, hexaflumuron, lufenuron, novaluron, teflubenzuron, triflumuron; buprofezin, diofenolan, hexythiazox, etoxazole, clofentazine; b) ecdysone antagonists such as halofenozide, methoxyfenozide, tebufenozide; c) juvenoids such as pyriproxyfen, methoprene, fenoxycarb; d) lipid biosynthesis inhibitors such as spirodiclofen;
neonicotinoids such as flonicamid, clothianidin, dinotefuran, imidacloprid, thiamethoxam, nitenpyram, nithiazin, acetamiprid, thiacloprid;
further unclassified insecticides such as abamectin, acequinocyl, acetamiprid, amitraz, azadirachtin, bensultap bifenazate, cartap, chlorfenapyr, chlordimeform, cyromazine, diafenthiuron, dinetofuran, diofenolan, emamectin, endosulfan, ethiprole, fenazaquin, fipronil, formetanate, formetanate hydrochloride, gamma-HCH hydramethylnon, imidacloprid, indoxacarb, isoprocarb, metolcarb, pyridaben, pymetrozine, spinosad, tebufenpyrad, thiamethoxam, thiocyclam, XMC and xylylcarb;
N-phenylsemicarbazones, as are described in EP-A 462 456 by the general formula I, in particular compounds of the general formula IV

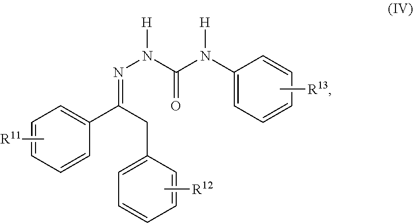

in which R¹¹ and R¹², independently of one another, are hydrogen, halogen, CN, C1-C4-alkyl, C1-C4-alkoxy, C1-C4-haloalkyl or C1-C4-haloalkoxy, and R¹³ is C1-C4-alkoxy, C1-C4-haloalkyl or C1-C4-haloalkoxy, e.g. compound IV in which R¹¹ is 3-CF3 and R¹² is 4-CN and R¹³ is 4-OCF₃.

Growth regulators which can be used are e.g. chlormequat chloride, mepiquat chloride, prohexadione-calcium or those from the group of gibberellins. These include, for example, the gibberellins GA1, GA3, GA4, GA5 and GA7 etc. and the corresponding exo-16,17-dihydrogibberellins, and also the derivatives thereof, e.g. the esters with C1-C4-carboxylic acids. According to the invention, preference is given to exo-16,17-dihydro-GA5 13-acetate.

A preferred embodiment of the invention relates to the use according to the invention of hydrophobin for the preparation of aqueous active ingredient compositions of fungicides, in particular strobilurins, azoles and 6-aryltriazolo[1,5a]pyrimidines, as are described e.g. in WO 98/46608, WO 99/41255 or WO 03/004465 in each case by the general formula I (page 1, line 8 to page 11, line 45, and also compounds depicted in formula IA in conjunction with tables 1 to 44 and table A in WO 03/00465), in particular for active ingredients of the general formula V,

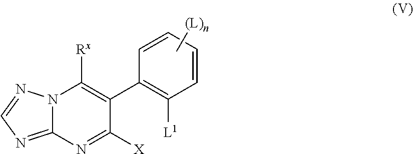

in which:
R^{x} is a group NR¹⁴R¹⁵, or linear or branched C1-C8-alkyl, which is optionally substituted by halogen, OH, C1-C4-alkoxy, phenyl or C3-C6-cycloalkyl, C2-C6-alkenyl, C3-C6-cycloalkyl, C3-C6-cycloalkenyl, phenyl or naphthyl, where the 4 last-mentioned radicals can have 1, 2, 3 or 4 substituents selected from halogen, OH, C1-C4-alkyl, C1-C4-haloalkoxy, C1-C4-alkoxy and C1-C4-haloalkyl;
R¹⁴, R¹⁵ independently of one another are hydrogen, C1-C8-alkyl, C1-C8-haloalkyl, C3-C10-cycloalkyl, C3-C6-halocycloalkyl, C2-C8-alkenyl, C4-C10-alkadienyl, C2-C8-haloalkenyl, C3-C6-cycloalkenyl, C2-C8-halocycloalkenyl, C2-C8-alkynyl, C2-C8-haloalkynyl or C3-C6-cycloalkynyl,
R¹⁴ and R¹⁵ together with the nitrogen atom to which they are bonded, are five- to eight-membered heterocyclyl, which is bonded via N and can comprise one, two or three further heteroatoms from the group O, N and S as ring member and/or can carry one or more substituents from the group consisting of halogen, C1-C6-alkyl, C1-C6-haloalkyl, C2-C6-alkenyl, C2-C6-haloalkenyl, C1-C6-alkoxy, C1-C6-haloalkoxy, C3-C6-alkenyloxy, C3-C6-haloalkenyloxy, (exo)-C1-C6-alkylene and oxy-C1-C3-alkyleneoxy;
L is selected from halogen, cyano, C1-C6-alkyl, C1-C4-haloalkyl, C1-C6-alkoxy, C1-C4-haloalkoxy and C1-C6-alkoxycarbonyl;
L¹ is halogen, C1-C6-alkyl or C1-C6-haloalkyl and in particular fluorine or chlorine;
X is halogen, C1-C4-alkyl, cyano, C1-C4-alkoxy or C1-C4-haloalkyl and is preferably halogen or methyl and in particular chlorine.

Examples of compounds of the formula V are
5-chloro-7-(4-methylpiperidin-1-yl)-6-(2,4,6-trifluorophenyl)-[1,2,4]triazolo[1,5-a]pyrimidine,
5-chloro-7-(4-methylpiperazin-1-yl)-6-(2,4,6-trifluorophenyl)-[1,2,4]triazolo[1,5-a]pyrimidine,
5-chloro-7-(morpholin-1-yl)-6-(2,4,6-trifluorophenyl)-[1,2,4]triazolo[1,5-a]pyrimidine,
5-chloro-7-(piperidin-1-yl)-6-(2,4,6-trifluorophenyl)-[1,2,4]triazolo[1,5-a]pyrimidine,
5-chloro-7-(morpholin-1-yl)-6-(2,4,6-trifluorophenyl)-[1,2,4]triazolo[1,5-a]pyrimidine,
5-chloro-7-(isopropylamino)-6-(2,4,6-trifluorophenyl)-[1,2,4]triazolo[1,5-a]pyrimidine,
5-chloro-7-(cyclopentylamino)-6-(2,4,6-trifluorophenyl)-[1,2,4]triazolo[1,5-a]pyrimidine,
5-chloro-7-(2,2,2-trifluoroethylamino)-6-(2,4,6-trifluorophenyl)-[1,2,4]triazolo[1,5-a]pyrimidine,
5-chloro-7-(1,1,1-trifluoropropan-2-ylamino)-6-(2,4,6-trifluorophenyl)-[1,2,4]triazolo[1,5-a]pyrimidine,
5-chloro-7-(3,3-dimethylbutan-2-ylamino)-6-(2,4,6-trifluorophenyl)-[1,2,4]triazolo[1,5-a]pyrimidine,
5-chloro-7-(cyclohexylmethyl)-6-(2,4,6-trifluorophenyl)-[1,2,4]triazolo[1,5-a]pyrimidine,
5-chloro-7-(cyclohexyl)-6-(2,4,6-trifluorophenyl)-[1,2,4]triazolo[1,5-a]pyrimidine,
5-chloro-7-(2-methylbutan-3-yl)-6-(2,4,6-trifluorophenyl)-[1,2,4]triazolo[1,5-a]pyrimidine,
5-chloro-7-(3-methylpropan-1-yl)-6-(2,4,6-trifluorophenyl)-[1,2,4]triazolo[1,5-a]pyrimidine,
5-chloro-7-(4-methylcyclohexan-1-yl)-6-(2,4,6-trifluorophenyl)-[1,2,4]triazolo[1,5-a]pyrimidine,
5-chloro-7-(hexan-3-yl)-6-(2,4,6-trifluorophenyl)-[1,2,4]triazolo[1,5-a]pyrimidine,
5-chloro-7-(2-methylbutan-1-yl)-6-(2,4,6-trifluorophenyl)-[1,2,4]triazolo[1,5-a]pyrimidine,
5-chloro-7-(3-methylbutan-1-yl)-6-(2,4,6-trifluorophenyl)-[1,2,4]triazolo[1,5-a]pyrimidine,
5-chloro-7-(1-methylpropan-1-yl)-6-(2,4,6-trifluorophenyl)-[1,2,4]triazolo[1,5-a]pyrimidine,
5-methyl-7-(4-methylpiperidin-1-yl)-6-(2,4,6-trifluorophenyl)-[1,2,4]triazolo[1,5-a]pyrimidine,
5-methyl-7-(4-methylpiperazin-1-yl)-6-(2,4,6-trifluorophenyl)-[1,2,4]triazolo[1,5-a]pyrimidine,
5-methyl-7-(morpholin-1-yl)-6-(2,4,6-trifluorophenyl)-[1,2,4]triazolo[1,5-a]pyrimidine,
5-methyl-7-(piperidin-1-yl)-6-(2,4,6-trifluorophenyl)-[1,2,4]triazolo[1,5-a]pyrimidine,
5-methyl-7-(morpholin-1-yl)-6-(2,4,6-trifluorophenyl)-[1,2,4]triazolo[1,5-a]pyrimidine,
5-methyl-7-(isopropylamino)-6-(2,4,6-trifluorophenyl)-[1,2,4]triazolo[1,5-a]pyrimidine,
5-methyl-7-(cyclopentylamino)-6-(2,4,6-trifluorophenyl)-[1,2,4]triazolo[1,5-a]pyrimidine,
5-methyl-7-(2,2,2-trifluoroethylamino)-6-(2,4,6-trifluorophenyl)-[1,2,4]triazolo[1,5-a]pyrimidine,
5-methyl-7-(1,1,1-trifluoropropan-2-ylamino)-6-(2,4,6-trifluorophenyl)-[1,2,4]triazolo[1,5-a]pyrimidine,
5-methyl-7-(3,3-dimethylbutan-2-ylamino)-6-(2,4,6-trifluorophenyl)-[1,2,4]triazolo[1,5-a]pyrimidine,
5-methyl-7-(cyclohexylmethyl)-6-(2,4,6-trifluorophenyl)-[1,2,4]triazolo[1,5-a]pyrimidine,
5-methyl-7-(cyclohexyl)-6-(2,4,6-trifluorophenyl)-[1,2,4]triazolo[1,5-a]pyrimidine,
5-methyl-7-(2-methylbutan-3-yl)-6-(2,4,6-trifluorophenyl)-[1,2,4]triazolo[1,5-a]pyrimidine, 5-methyl-7-(3-methylpropan-1-yl)-6-(2,4,6-trifluorophenyl)-[1,2,4]triazolo[1,5-a]pyrimidine,
5-methyl-7-(4-methylcyclohexan-1-yl)-6-(2,4,6-trifluorophenyl)-[1,2,4]triazolo[1,5-a]pyrimidine,
5-methyl-7-(hexan-3-yl)-6-(2,4,6-trifluorophenyl)-[1,2,4]triazolo[1,5-a]pyrimidine,
5-methyl-7-(2-methylbutan-1-yl)-6-(2,4,6-trifluorophenyl)-[1,2,4]triazolo[1,5-a]pyrimidine, 5-methyl-7-(3-methylbutan-1-yl)-6-(2,4,6-trifluorophenyl)-[1,2,4]triazolo[1,5-a]pyrimidine and
5-methyl-7-(1-methylpropan-1-yl)-6-(2,4,6-trifluorophenyl)-[1,2,4]triazolo[1,5-a]pyrimidine.

Individual embodiments of the present invention are described in detail by the following examples. These examples merely serve to illustrate the invention and must not be interpreted as being a limitation of the subject matter of the invention.

### EXAMPLES

Unless stated otherwise, all of the reagents used were of the highest possible commercially available, purity, and all commercially available reagents, instruments, antibodies and kits were used in accordance with the manufacturer's instructions.

The hydrophobins used in the examples were prepared according to the examples from part A of WO 2007/014897.

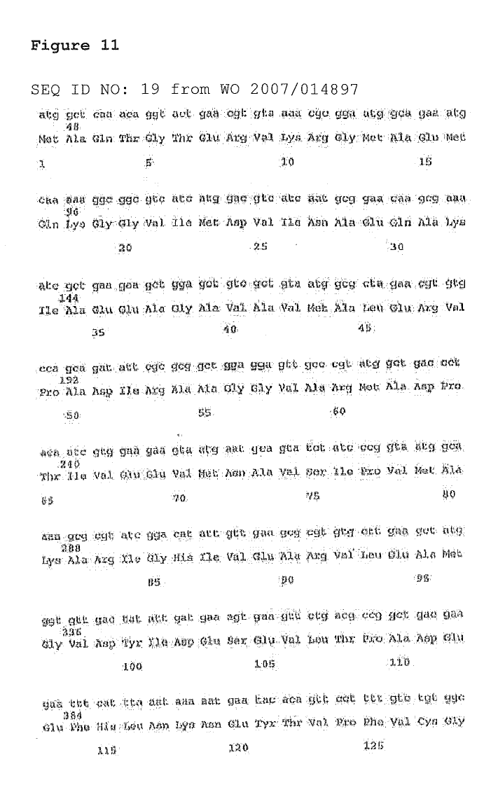

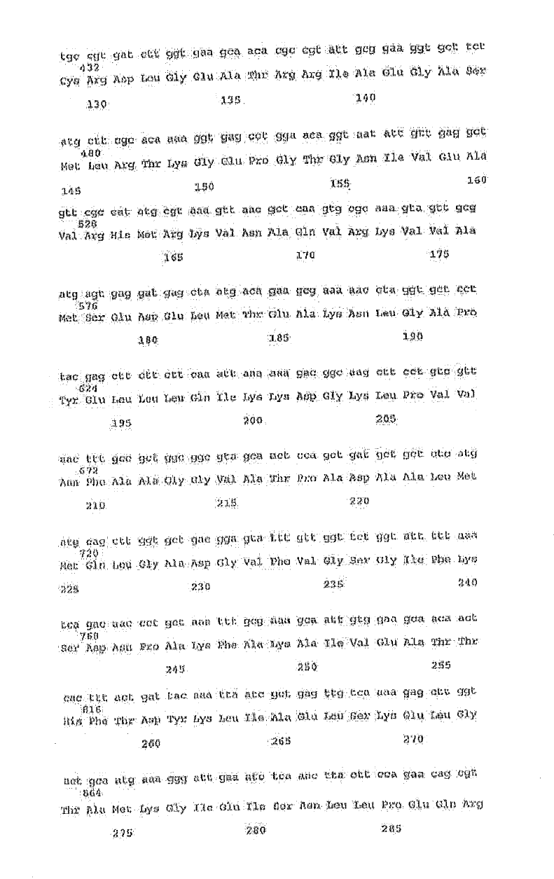

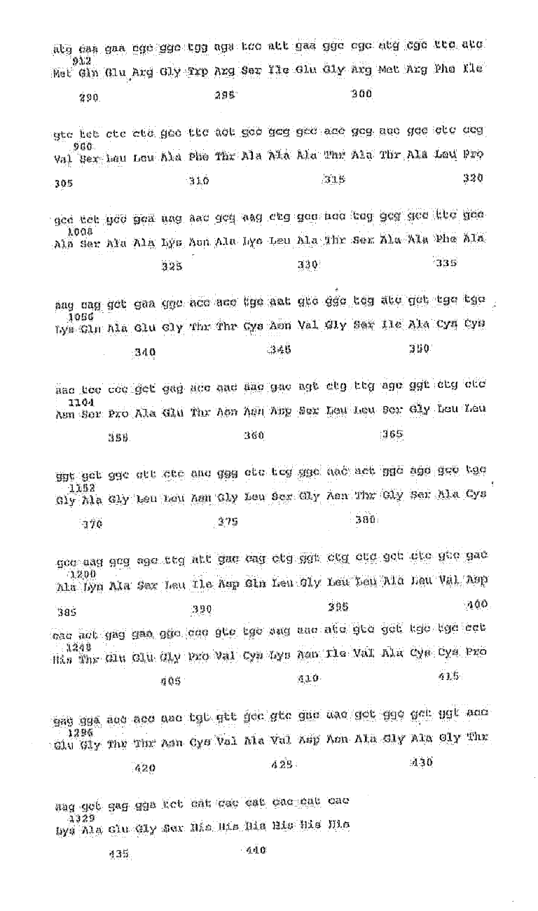

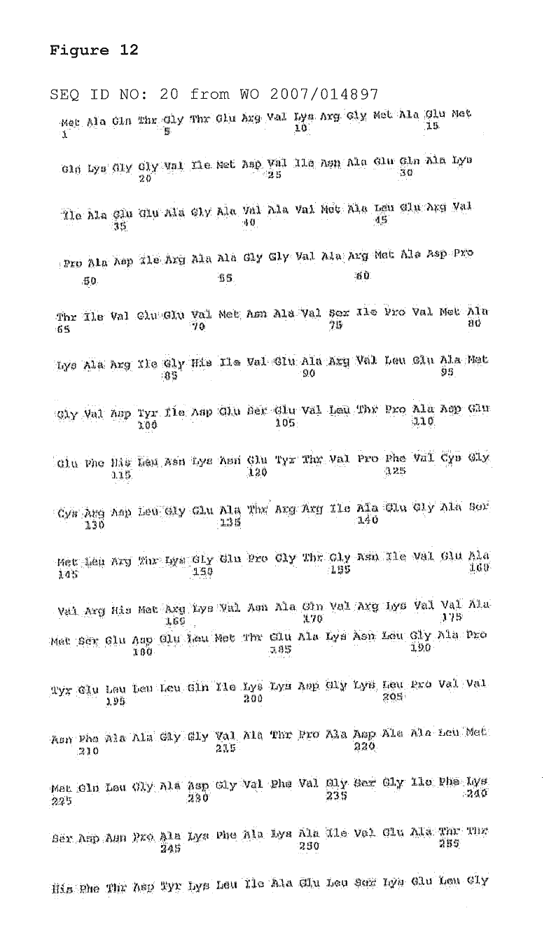

The sequence of the “hydrophobin A” used in the examples is given in WO 2007/014897 under SEQ ID NO:19 and 20 (FIGS. 11 and 12 of this application). This “hydrophobin A” is the hydrophobin dewA which has been fused with the protein yaad. Furthermore, the construct also comprises an Xa cleavage site and a His tag (yaad-Xa-dewA-his).

The sequence of the “hydrophobin B” used in the examples is given in WO 2007/014897 under SEQ ID NO:25 and 26 (FIGS. 13 and 14 of this application). This “hydrophobin B” is the hydrophobin dewA which has been fused with a shortened protein yaad. Furthermore, the construct also comprises an Xa cleavage site and a His tag (yaad40-Xa-dewA-his).

### EXAMPLE 1

### Binding to Skin 1 (Qualitative)

A visual qualitative test was developed in order to examine whether hydrophobin binds to skin.

### Solutions Used:

Blocking solution: DIG Wash+Buffer set 1585762 Boehringer MA (10× solution) diluted in TBS
TBS: 20 mM Tris; 150 mM NaCl pH 7.5
TTBS: TBS+0.05% Tween 20

The first step is the transfer of the external keratin layer from the skin onto a stable carrier. For this, a transparent adhesive strip was applied firmly to depilated human skin and removed again. The test can be carried out directly on the transparent adhesive strip or the adhering keratin layer can be transferred to a glass slide by sticking once again. The demonstration of binding was undertaken as follows:
for incubation with the various reagents, transfer to a Falcon vessel
optionally addition of ethanol for degreasing, removal of ethanol and drying of the slide
incubation for 1 h at room temperature with blocking buffer
washing for 2×5 min with TTBS
washing for 1×5 min with TBS
incubation with the hydrophobin to be tested (coupled to tag—e.g. His₆, Ha etc.) or control protein in TBS/0.05% Tween 20 for 2-4 h at room temperature
removal of the supernatant
3× washing with TBS
incubation for 1 h at room temperature with monoclonal anti-poly-histidine antibodies, diluted 1:2000 in TBS+0.01% blocking
washing for 2×5 min with TTBS
washing for 1×5 min with TBS
incubation for 1 h at room temperature with anti-mouse IgG alkaline phosphatase conjugate, diluted 1:5000 in TBS+0.01% blocking
washing for 2×5 min with TTBS
washing for 1×5 min with TBS
addition of phosphatase substrate (NBT-BCIP; Boehringer MA 1 tablet/40 ml of water 2.5 min; stop: with water)
optical detection of the colored precipitate with the naked eye or using a microscope. A blue colored precipitate indicates that hydrophobia has bonded to the skin.

The binding to nails can be ascertained analogously by incubating the hydrophobins to be investigated directly with the nail surface and measuring accordingly.

### EXAMPLE 2

### Binding to Skin 2 (Quantitative)

A quantitative test was developed which allows the skin binding strength of the hydrophobin to be compared with non-specific proteins (FIG. 2).

A 5 mm cork borer was used to bore out a section from a thawed dry piece of skin without hair (human or pig) (or in the case of a surface test a section of skin was inserted into a Falcon lid). The skin sample was then converted to a thickness of 2-3 mm in order to remove any tissue present. The skin sample was then transferred to an Eppendorf vessel (protein low-bind) in order to carry out the binding demonstration (see also FIG. 2):
2× washing with PBS/0.05% Tween 20
addition of 1 ml of 1% BSA in PBS and incubation for 1 h at room temperature, gentle swinging movements (900 rpm)
removal of the supernatant
addition of 100 μg of hydrophobin in PBS+0.05% Tween 20; incubation for 2 h at room temperature and gentle swinging movements (900 rpm)
removal'of the supernatant
3× washing with PBS/0.05% Tween 20
incubation with 1 ml of monoclonal mouse anti-tag-His₆ or HA-specific antibodies with peroxidase conjugate (1:2000 in PBS/0.05% Tween 20) [Monoclonal AntipolyHistidine Peroxidase Conjugate, produced in mouse, lyophilized powder, Sigma] for 2-4 h at room temperature, gentle swinging movement (900 rpm)
3× washing with PBS/0.05% Tween 20
addition of peroxidase substrate (1 ml/Eppendorf vessel; composition see below)
allow reaction to proceed until a blue coloration is obtained (about 1:30 minutes).
Stop the reaction with 100 μl of 2M H₂SO₄.
The absorption was measured at 405 nm.

### Peroxidase Substrate (Prepare Shortly Beforehand):

0.1 ml of TMB solution (42 mM TMB in DMSO)+10 ml of substrate buffer (0.1M sodium acetate pH 4.9)+14.7 μl H₂O₂ 3% strength

An increase in the absorption compared with a sample without hydrophobin indicates that hydrophobin has bonded to the skin. See ex. 3 for the background.

The binding to mucous membrane can also be measured analogously by taking a sample from mucous membrane (for example human mouth mucous membrane) by means of a transparent adhesive strip, which can then be investigated as to binding effect.

### EXAMPLE 3

### Binding to Hair (Quantitative)

In order to be able to demonstrate the binding strength of the hydrophobin to hair also compared to other proteins, a quantitative assay was developed (FIG. 2 in WO 2006/136607). In this test, hair was firstly incubated with hydrophobin and excess hydrophobin was washed off. An antibody-peroxidase conjugate was then coupled via the His tag of the hydrophobin. Non-bonded antibody-peroxidase conjugate was washed off again. The bonded antibody-peroxidase conjugate [Monoclonal AntipolyHistidine Peroxidase Conjugate, produced in mouse, lyophilized powder, Sigma] can convert a colorless substrate (TMB) to a colored product, which is measured phometrically at 405 nm. The intensity of the absorption indicates the amount of bonded hydrophobin or comparison protein. The comparison protein selected was e.g. yaad from B. subtilis, which likewise had—as is necessary for this test—a His tag for detection. Instead of the His tag it is also possible to use other specific antibodies conjugated with peroxidase.

5 mg of hair (human) are cut into sections 5 mm in length and transferred to Eppendorf vessels (protein low-bind) in order to carry out the binding demonstration:
addition of 1 ml of ethanol for degreasing
centrifugation, removal of ethanol and washing of the hair with H₂O
addition of 1 ml of 1% BSA in PBS and incubation for 1 h at room temperature, gentle swinging movements
centrifugation, removal of the supernatant
addition of the hydrophobin to be tested (coupled to tag—e.g. His₆, HA etc.) or control protein in 1 ml of PBS/0.05% Tween 20; incubation for 16 h at 4° C. (or at least 2 h at room temperature) with gentle swinging movements.
Centrifugation, removal of the supernatant
3× washing with PBS/0.05% Tween 20
incubation with 1 ml of monoclonal mouse anti-tag (His₆ or HA) antibodies with peroxidase conjugate (1:2000 in PBS/0.05% Tween 20) [Monoclonal AntipolyHistidine Peroxidase Conjugate, produced in mouse, lyophilized powder, Sigma] for 2-4 h at room temperature, gentle swinging movement
3× washing with PBS/0.05% Tween 20
addition of peroxidase substrate (1 ml/Eppendorf vessel)
allow the reaction to proceed until a blue coloration is obtained (ca. 2 minutes).
Stop the reaction with 100 μl of 2M H₂SO₄.
The absorption is measured at 405 nm

### Peroxidase Substrate (Prepare Shortly Beforehand):

0.1 ml TMB solution (42 mM TMB in DMSO)+10 ml of substrate buffer (0.1M sodium acetate pH 4.9)+14.7 μl H₂O₂ 3% strength
BSA=bovine serum albumin
PBS=phosphate buffered salt solution
   Tween 20=polyoxyethylene sorbitan monolaurate,
   n ca. 20
   TMB=3,5,3′,5′-tetramethylbenzidine

A binding test on hair carried out by way of example for hydrophobin demonstrated considerable superiority of the binding of hydrophobin to hair compared with significantly poorer binding of the comparison protein yaad:

TABLE 1
Quantitative hydrophobin activity test on hair:

| | | |
|---|---|---|
| 1 | Buffer | A_{405 nm} = 0.05 |
| 2 | Comparison protein yaad | A_{405 nm} = 0.12 |
| 3 | Hydrophobin | A_{405 nm} = 1.43 |

1) buffer;
2) comparison protein yaad;
3) hydrophobin. The table shows the measured absorption values at 405 nm.

### EXAMPLE 4

### Derivatization of Hydrophobin with “Alexa” Dye and Binding to Hair

A coupling of dyes to hydrophobins can take place via the SH groups of the cysteines. Prior to coupling the dye Alexa Fluor 532, the disulfide bridges of the hydrophobin are cleaved:
1 mg hydrophobin
0.5 ml buffer (75 mM Tris pH 8.0
   2.5 mM EDTA
   1 mM DTT)
Incubation for 30 minutes at 37° C.

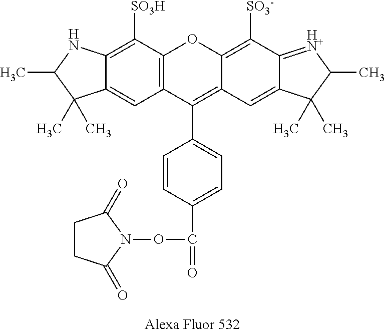

The coupling of the dye takes place in accordance with manufacturer's instructions (Alexa 532 Protein Labeling Kit; Molecular Probes; MP-A-10236).

The coating of human hair with Alexa-coupled hydrophobin is carried out as follows:
Incubate 10 mg of human hair with 50 μg/ml of Alexa hydrophobin or control protein yaad or uncoupled dye Alexa 532 in buffer TBS for 24 hours at room temperature
2× washing with TBS/0.05% Tween 20
1× washing with TBS
1× washing with TBS/1% SDS
detection in a fluorescence microscope (FIG. 4 of WO 2006/136607)

### EXAMPLE 5

### Test for Ascertaining the Spreading Behavior

In this test, the spreading behavior of other substances can be assessed against paraffin oil (thin-liquid) as reference substance.

A glass plate (20 cm×6.5 cm×0.2 cm) was wrapped with 2 strips of filter paper (type: Pörringer 1243/90) having a length of ca. 17 cm and width 1 cm. Wooden sticks with a diameter of 0.2 cm were used as spacers with respect to the glass plate. Here, it should be ensured that the measurement is not influenced by contact between the filter paper and the glass plate.

Using a pipette (direct displacement), 50 μl of test substance (e.g. the reference substance paraffin oil or the test substances Luvitol or Dimethicone, all in pure form) were applied to the filter paper strips in the middle. After 5 minutes, the length of the spreading distance of the test substance on the filter paper, starting from the point of application was measured.

### REFERENCE EXAMPLES

Results of the length measurement from the starting point [mm]:

| | |
|---|---|
| Paraffin oil (thin-liquid) | 36 |
| Luvitol Lite (INCI: hydrogen polyisobutene) | 64 |
| Dimethicone | 22 |

Taking paraffin oil (thin-liquid) as reference substance with a value of 1 gives rise to the following relative spreading values:
Luvitol Lite: 1.78
Dimethicone: 0.61

Values above 1 correspond to better spreading behavior than paraffin oil (thin-liquid) and values below 1 correspond to poorer spreading behavior than paraffin oil (thin-liquid).

### APPLICATION EXAMPLES WITH HYDROPHOBIN

In order to test an improved spreading effect as a result of hydrophobin, the paper strips are incubated overnight in an aqueous 0.1% (w/v) hydrophobin B solution at room temperature and are then dried. The spreading behavior of paraffin (thin-liquid) is then assessed on these treated strips in relation to untreated filter paper.

Upon carrying out this test, it was found that hydrophobin increased the run distances of the test substances stated above by up to 10%, i.e. improves their flow properties—the spreading behavior.

A comparable measurement on pig skin produces correlating results like the filter paper method.

Instead of treating the test surface with hydrophobin, 0.1% hydrophobin (end concentration) can also be added as component to the test substance and be emulsified. In this case as well, extended run stretches of the test substances on the described surfaces are found.

1. The use of hydrophobin as a spreading agent in a composition for treating surfaces.

2. The use according to claim 1, where the hydrophobin is a hydrophobin of the structural formula (I).

3. The use according to claim 1, where the composition has an oil-body concentration of from 0 to 20% by weight.

4. The use according to claim 1, where the composition is a cosmetic or pharmaceutical composition.

5. The use according to claim 1, where the surface is a human or animal body surface.

6. The use according to claim 4, where the surface is skin.

7. The use according to claim 1, where the composition is a crop protection composition.

8. The use according to claim 1, where the surface is a surface made of plant material.

9. The use according to claim 8, where the surface is a plant surface.

10. The use according to claim 8, where the surface is cellulose-containing and preferably paper.

11. The use according to claim 1, where the composition comprises hydrophobin in a concentration of from 0.001 to 10% by weight.

12. The use according to claim 1, wherein the hydrophobin is selected from the group consisting of hydrophobins of the type dewA, rodA, hypA, hypB, sc3, basf1 and basf2, and is preferably a hydrophobin of the type dewA, hypA or hypB.

13. The use according to claim 1, where the hydrophobin is a constituent of a fusion protein, and where a fusion partner is preferably yaad (SEQ ID NO:16 in WO 2007/014897) or is a shortened yaad.

14. The use according to claim 13, where the hydrophobin is selected from the group consisting of yaad-Xa-dewA-his (SEQ ID NO:20 in WO 2006/082251), yaad-Xa-rodA-his (SEQ ID NO:22 in WO 2006/082251), yaad-Xa-basf1-his (SEQ ID NO:24 in WO 2006/082251) and from hydrophobins derived by truncating the yaad fusion partner (SEQ ID NO:16 in WO 2006/082251), in particular yaad40-Xa-dewA-his (SEQ ID NO:26 in WO 2007/014897).

15-17. (canceled)

18. A composition for surface treatment which comprises at least one hydrophobin as defined in claim 1.

19. The composition according to claim 18, which is non-greasy.

20. The composition according to claim 18, which is a cosmetic or pharmaceutical composition.

21. The composition according to claim 20, additionally comprising at least one cosmetically or pharmaceutically active ingredient, the absorption and/or effect of which is improved by the presence of hydrophobin.

22. The composition according to claim 18, which is a crop protection composition.

23. The composition according to claim 22, additionally comprising at least one crop protection active ingredient, the absorption and/or effect of which is improved by the presence of hydrophobin.


SEQUENCE LISTING (30 sequences)

SEQ ID NO: 1 - protein, 294 aa, Unknown
  basf-yaad
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGW

SEQ ID NO: 2 - protein, 197 aa, Unknown
  basf-yaae
MGLTIGVLGLQGAVREHIHAIEACGAAGLVVKRPEQLNEVDGLILPGGESTTMRRLIDTYQFMEPLREFAAQGKPMFGTCAGLIILAKEIAGSDNPHLGLLNVVVERNSFGRQVDSFEADLTIKGLDEPFTGVFIRAPHILEAGENVEVLSEHNGRIVAAKQGQFLGCSFHPELTEDHRVTQLFVEMVEEYKQKALV

SEQ ID NO: 3 - DNA, 1329 bp, Artificial Sequence
  basf-yaad-Xa-dewA-his
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatccattgaaggccgcatgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgagggatctcatcaccatcaccatcac

SEQ ID NO: 4 - protein, 443 aa, Artificial Sequence
  basf-yaad-Xa-dewA-his
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAEGSHHHHHH

SEQ ID NO: 5 - DNA, 1395 bp, Artificial Sequence
  basf-yaad-Xa-rodA-his
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatctattgaaggccgcatgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctcggatctcatcaccatcaccatcac

SEQ ID NO: 6 - protein, 465 aa, Artificial Sequence
  basf-yaad-Xa-rodA-his
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSILGSHHHHHH

SEQ ID NO: 7 - DNA, 1407 bp, Artificial Sequence
  basf-yaad-Xa-BASF1-his
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctggagatctattgaaggccgcatgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcctcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccaggtcaacaagcagtgcaagcagaacatcgcctgctgccagaactccccttccgacgccaccggctccctcgtcaacctcggcctcggcaacccttgcatccctgtctccctcctccatatgggatctcatcaccatcaccatcac

SEQ ID NO: 8 - protein, 469 aa, Artificial Sequence
  basf-yaad-Xa-BASF1-his
MAQTGTERVKRGMAEMQKGGVIMDVINAEQAKIAEEAGAVAVMALERVPADIRAAGGVARMADPTIVEEVMNAVSIPVMAKARIGHIVEARVLEAMGVDYIDESEVLTPADEEFHLNKNEYTVPFVCGCRDLGEATRRIAEGASMLRTKGEPGTGNIVEAVRHMRKVNAQVRKVVAMSEDELMTEAKNLGAPYELLLQIKKDGKLPVVNFAAGGVATPADAALMMQLGADGVFVGSGIFKSDNPAKFAKAIVEATTHFTDYKLIAELSKELGTAMKGIEISNLLPEQRMQERGWRSIEGRMKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHMGSHHHHHH

SEQ ID NO: 9 - DNA, 882 bp, Bacillus subtilis
atggctcaaacaggtactgaacgtgtaaaacgcggaatggcagaaatgcaaaaaggcggcgtcatcatggacgtcatcaatgcggaacaagcgaaaatcgctgaagaagctggagctgtcgctgtaatggcgctagaacgtgtgccagcagatattcgcgcggctggaggagttgcccgtatggctgaccctacaatcgtggaagaagtaatgaatgcagtatctatcccggtaatggcaaaagcgcgtatcggacatattgttgaagcgcgtgtgcttgaagctatgggtgttgactatattgatgaaagtgaagttctgacgccggctgacgaagaatttcatttaaataaaaatgaatacacagttccttttgtctgtggctgccgtgatcttggtgaagcaacacgccgtattgcggaaggtgcttctatgcttcgcacaaaaggtgagcctggaacaggtaatattgttgaggctgttcgccatatgcgtaaagttaacgctcaagtgcgcaaagtagttgcgatgagtgaggatgagctaatgacagaagcgaaaaacctaggtgctccttacgagcttcttcttcaaattaaaaaagacggcaagcttcctgtcgttaactttgccgctggcggcgtagcaactccagctgatgctgctctcatgatgcagcttggtgctgacggagtatttgttggttctggtatttttaaatcagacaaccctgctaaatttgcgaaagcaattgtggaagcaacaactcactttactgattacaaattaatcgctgagttgtcaaaagagcttggtactgcaatgaaagggattgaaatctcaaacttacttccagaacagcgtatgcaagaacgcggctgg

SEQ ID NO: 10 - protein, 294 aa, Bacillus subtilis; identical to SEQ ID NO: 1

SEQ ID NO: 11 - DNA, 1329 bp, Artificial Sequence; identical to SEQ ID NO: 3
  fusion protein

SEQ ID NO: 12 - protein, 443 aa, Artificial Sequence; identical to SEQ ID NO: 4
  fusion protein

SEQ ID NO: 13 - DNA, 561 bp, Artificial Sequence
  fusion protein
atggctcaaacaggtactgaacgtgtaaaacgcggaatggcagaaatgcaaaaaggcggcgtcatcatggacgtcatcaatgcggaacaagcgaaaatcgctgaagaagctggagctgtcattgaaggccgcatgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgagggatctcatcaccatcaccatcac

SEQ ID NO: 14 - protein, 187 aa, Artificial Sequence
  fusion protein
MAQTGTERVKRGMAEMQKGGVIMDVINAEQAKIAEEAGAVIEGRMRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAEGSHHHHHH

SEQ ID NO: 15 - DNA, 405 bp, Unknown
  basf-dewA
atgcgcttcatcgtctctctcctcgccttcactgccgcggccaccgcgaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgctggtaccaaggctgag

SEQ ID NO: 16 - protein, 135 aa, Unknown
  basf-dewA
MRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAE

SEQ ID NO: 17 - DNA, 471 bp, Unknown
  basf-rodA
atgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctc

SEQ ID NO: 18 - protein, 157 aa, Unknown
  basf-rodA
MKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSIL

SEQ ID NO: 19 - DNA, 336 bp, Unknown
  basf-HypA
atgatctctcgcgtccttgtcgctgctctcgtcgctctccccgctcttgttactgcaactcctgctcccggaaagcctaaagccagcagtcagtgcgacgtcggtgaaatccattgctgtgacactcagcagactcccgaccacaccagcgccgccgcgtctggtttgcttggtgttcccatcaaccttggtgctttcctcggtttcgactgtacccccatttccgtccttggcgtcggtggcaacaactgtgctgctcagcctgtctgctgcacaggaaatcaattcaccgcattgattaacgctcttgactgctctcctgtcaatgtcaacctc

SEQ ID NO: 20 - protein, 112 aa, Unknown
  basf-HypA
MISRVLVAALVALPALVTATPAPGKPKASSQCDVGEIHCCDTQQTPDHTSAAASGLLGVPINLGAFLGFDCTPISVLGVGGNNCAAQPVCCTGNQFTALINALDCSPVNVNL

SEQ ID NO: 21 - DNA, 357 bp, Unknown
  basf-HypB
atggtcagcacgttcatcactgtcgcaaagacccttctcgtcgcgctcctcttcgtcaatatcaatatcgtcgttggtactgcaactaccggcaagcattgtagcaccggtcctatcgagtgctgcaagcaggtcatggattctaagagccctcaggctacggagcttcttacgaagaatggccttggcctgggtgtccttgctggcgtgaagggtcttgttggcgcgaattgcagccctatcacggcaattggtattggctccggcagccaatgctctggccagaccgtttgctgccagaataataatttcaacggtgttgtcgctattggttgcactcccattaatgccaatgtg

SEQ ID NO: 22 - protein, 119 aa, Unknown
  basf-HypB
MVSTFITVAKTLLVALLFVNINIVVGTATTGKHCSTGPIECCKQVMDSKSPQATELLTKNGLGLGVLAGVKGLVGANCSPITAIGIGSGSQCSGQTVCCQNNNFNGVVAIGCTPINANV

SEQ ID NO: 23 - DNA, 408 bp, Unknown
  basf-sc3
atgttcgcccgtctccccgtcgtgttcctctacgccttcgtcgcgttcggcgccctcgtcgctgccctcccaggtggccacccgggcacgaccacgccgccggttacgacgacggtgacggtgaccacgccgccctcgacgacgaccatcgccgccggtggcacgtgtactacggggtcgctctcttgctgcaaccaggttcaatcggcgagcagcagccctgttaccgccctcctcggcctgctcggcattgtcctcagcgacctcaacgttctcgttggcatcagctgctctcccctcactgtcatcggtgtcggaggcagcggctgttcggcgcagaccgtctgctgcgaaaacacccaattcaacgggctgatcaacatcggttgcacccccatcaacatcctc

SEQ ID NO: 24 - protein, 136 aa, Unknown
  basf-sc3
MFARLPVVFLYAFVAFGALVAALPGGHPGTTTPPVTTTVTVTTPPSTTTIAAGGTCTTGSLSCCNQVQSASSSPVTALLGLLGIVLSDLNVLVGISCSPLTVIGVGGSGCSAQTVCCENTQFNGLINIGCTPINIL

SEQ ID NO: 25 - DNA, 483 bp, Artificial Sequence
  basf-BASF1
atgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcctcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccaggtcaacaagcagtgcaagcagaacatcgcctgctgccagaactccccttccgacgccaccggctccctcgtcaacctcggcctcggcaacccttgcatccctgtctccctcctccatatg

SEQ ID NO: 26 - protein, 161 aa, Artificial Sequence
  basf-BASF1
MKFSVSAAVLAFAASVAALPQHDSAAGNGNGVGNKFPVPDDVTVKQATDKCGDQAQLSCCNKATYAGDVLTDIDEGILAGLLKNLIGGGSGSEGLGLFDQCVKLDLQISVIGIPIQDLLNQVNKQCKQNIACCQNSPSDATGSLVNLGLGNPCIPVSLLHM

SEQ ID NO: 27 - DNA, 465 bp, Artificial Sequence
  basf-BASF2
atgaagttctccgtctccgccgccgtcctcgccttcgccgcctccgtcgccgccctccctcagcacgactccgccgccggcaacggcaacggcgtcggcaacaagttccctgtccctgacgacgtcaccgtcaagcaggccaccgacaagtgcggcgaccaggcccagctctcctgctgcaacaaggccacctacgccggcgacgtcaccgacatcgacgagggcatcctcgccggcctcctcaagaacctcatcggcggcggctccggctccgagggcctcggcctcttcgaccagtgcgtcaagctcgacctccagatctccgtcatcggcatccctatccaggacctcctcaaccagcagtgcaagcagaacatcgcctgctgccagaactccccttccgacgccaccggctccctcgtcaacctcggcaacccttgcatccctgtctccctcctccatatg

SEQ ID NO: 28 - protein, 155 aa, Artificial Sequence
  basf-BASF2
MKFSVSAAVLAFAASVAALPQHDSAAGNGNGVGNKFPVPDDVTVKQATDKCGDQAQLSCCNKATYAGDVTDIDEGILAGLLKNLIGGGSGSEGLGLFDQCVKLDLQISVIGIPIQDLLNQQCKQNIACCQNSPSDATGSLVNLGNPCIPVSLLHM

SEQ ID NO: 29 - DNA, 882 bp, Unknown; identical to SEQ ID NO: 9
  basf-yaad

SEQ ID NO: 30 - DNA, 591 bp, Unknown
  basf-yaae
atgggattaacaataggtgtactaggacttcaaggagcagttagagagcacatccatgcgattgaagcatgcggcgcggctggtcttgtcgtaaaacgtccggagcagctgaacgaagttgacgggttgattttgccgggcggtgagagcacgacgatgcgccgtttgatcgatacgtatcaattcatggagccgcttcgtgaattcgctgctcagggcaaaccgatgtttggaacatgtgccggattaattatattagcaaaagaaattgccggttcagataatcctcatttaggtcttctgaatgtggttgtagaacgtaattcatttggccggcaggttgacagctttgaagctgatttaacaattaaaggcttggacgagccttttactggggtattcatccgtgctccgcatattttagaagctggtgaaaatgttgaagttctatcggagcataatggtcgtattgtagccgcgaaacaggggcaattccttggctgctcattccatccggagctgacagaagatcaccgagtgacgcagctgtttgttgaaatggttgaggaatataagcaaaaggcacttgta